(19) United States
(12) Patent Application Publication
(10) Pub. No.: US 2002/0042122 A1
(43) Pub. Date: Apr. 11, 2002

(54) Mammalian proteins; related reagents and methods

(76) Inventor: J. Fernando Bazan, Palo Alto, CA (US)

Correspondence Address: DNAX RESEARCH INSTITUTE, LEGAL DEPARTMENT, 901 CALIFORNIA AVENUE, PALO ALTO, CA, 94304, US

(21) Appl. No.: 09/745,003

(22) Filed: Dec. 20, 2000

Related U.S. Application Data
(60) Provisional application No. 60/172,090, filed on Dec. 23, 1999.

(51) Int. Cl. A61K 39/00; A61K 39/38; C12N 1/20; C07K 1/00; C07K 14/00; C07K 17/00

(52) U.S. Cl. 435/252.3; 530/350; 424/184.1

(57) ABSTRACT

Mammalian polypeptides, isolated proteins, and fragments thereof including the polynucleotides encoding them. Antibodies, both polyclonal and monoclonal, are also provided. Methods of using the compositions for both diagnostic and therapeutic utilities are provided.

This filing claims benefit of U.S. Provisional Patent application U.S. Ser. No. 60/172,090, filed Dec. 23, 1999.

### FIELD OF THE INVENTION

The present invention relates to compositions and methods for affecting mammalian physiology, including specific organ functions. In particular, it provides polynucleotides, polypeptides, and antibodies that are related to compounds associated with mammalian degenerative disease. Diagnostic and therapeutic uses of these materials are also disclosed.

### BACKGROUND OF THE INVENTION

Prions are proteinaceous compositions suspected of acting as infectious pathogens in mammalian central nervous system disease. A major advance in the study of neurodegenerative disease was the discovery and purification of a protein designated prion protein (PrP). Bolton, et al. (1982) Science 218:1309-11; Prusiner, et al. (1982) Biochemistry 21:6942-50; and McKinley, et al. (1983) Cell 35:57-62.

The PrP protein is encoded by a single-copy host gene that is believed to be normally expressed in a soluble, disease-free form designated PrP^{c}. Basler, et al. (1986) Cell 46:417-28. However, PrP^{c} can be transformed into an insoluble, disease-associated form designated PrP^{Sc}—named for its association with the neurodegenerative disease of sheep and goats called scrapie.

The insoluble PrP^{Sc} isoform has been demonstrated to be required for both the transmission and pathogenicity of certain mammalian neurodegenerative diseases. Prusiner (1991) Science 252:1515-1522. In particular, the human diseases: Kuru, Creutzfeldt-Jakob Disease (CJD), Gerstmann-Straussler-Scheinker Disease (GSS), and fatal familial insomnia (FFI) are all suspected of being prion-associated diseases caused by insoluble PrP^{Sc} isoforms. Gajdusek (1977) Science 197:943-960; and Medori, et al. (1992) N. Engl. J. Med. 326:444-449.

Accordingly, there exists a need for the discovery and development of additional prion-like compositions similar to PrPs. Such a discovery will further our understanding of prion-like compositions in normal and disease states and permit development of new therapies to ameliorate associated abnormal conditions. The present provides new mammalian prion-like compositions designated PrP2s, related compositions, e.g., genes and antibodies, and methods for their research, diagnostic, or therapeutic use.

### SUMMARY OF THE INVENTION

The present invention is directed to novel polypeptides, e.g., primate or rodent, and molecular structures designated PrP2, and their biological activities. It encompasses polynucleotides encoding the polypeptides themselves and methods for their production and use. The polynucleotides of the invention are characterized, in part, by their homology to complementary DNA (cDNA) sequences enclosed herein.

In certain embodiments, the invention provides an isolated, or recombinant PrP2 comprising a mature polypeptide sequence of SEQ ID NO: 2, 4, or 6, or antigenic fragments thereof at least 12 contiguous amino acid residues in length; or a polypeptide comprising an antigenic PrP2 fragment of SEQ ID NO: 2, 4, 6, wherein the length of the fragment of contiguous amino acid residues is at least 15, 22, 25, 30, 40, 50, 75, or 100 contiguous residues in number; a soluble PrP2^{c} polypeptide; an insoluble PrP2^{Sc}; and a fusion polypeptide comprising PrP2 sequence of SEQ ID NO: 2, 4, or 6.

In further polypeptide embodiments, the invention provides: a PrP2 comprising a mature sequence of or SEQ ID NO: 2, 4, or 6; a polypeptide that specifically binds antibody raised against a polypeptide comprising said PrP2 polypeptide sequence; or the polypeptide: is from a warm blooded animal, e.g., a primate, such as a human; comprises at least one polypeptide segment of SEQ ID NO: 2, 4, or 6; comprises a plurality of portions exhibiting the identity; comprises a plurality of polypeptide segments of SEQ ID NO: 2, 4, or 6; is a natural allelic variant of a primate or rodent PrP2; has a length of at least about 120 amino acids; exhibits at least two non-overlapping epitopes specific for a primate or rodent PrP2; exhibits a sequence identity over a length of at least about 35 amino acids to a primate or rodent PrP2; comprises heterologous amino acid sequence; is encoded by polynucleotide sequence operably linked in an expression vector; is a full length mature polypeptide of SEQ ID NO: 2, 4, or 6; is a synthetic polypeptide; is attached to a solid substrate; is conjugated to another chemical moiety; is a 5-fold or less substitute of natural sequence; or is a deletion or insertion variant from a natural sequence. Certain preferred embodiments include compositions comprising: a sterile PrP2 polypeptide; a soluble PrP2^{c}; an insoluble PrP2^{Sc}; or any combination of the above wherein the PrP2 polypeptide is with a carrier, wherein the carrier is: an aqueous compound, including water, saline, and/or buffer; and/or formulated for oral, rectal, nasal, topical, or parenteral administration; a sterile polypeptide; or the polypeptide, as described, and a carrier, wherein the carrier is: an aqueous compound, including water, saline, and/or buffer; and/or formulated for oral, rectal, nasal, topical, or parenteral administration.

Certain fusion polypeptides are also provided, e.g., comprising: mature polypeptide sequence of SEQ ID NO: 2, 4, or 6; a detection or purification tag, including a FLAG, His6, or Ig sequence; or sequence of another polypeptide. Kit embodiments include a kit comprising such a polypeptide, and: a compartment comprising the polypeptide; and/or instructions for use or disposal of reagents in the kit.

The invention also provides binding compound embodiments comprising an antigen binding site from an antibody, that specifically and/or selectively binds PrP2, wherein: the PrP2 is a primate or rodent polypeptide; the binding compound is an Fv, Fab, or Fab2 fragment; the binding compound is conjugated to another chemical moiety; or the antibody: is raised against a polypeptide sequence of a mature polypeptide comprising PrP2 sequence of SEQ ID NO: 2, 4, or 6; is raised against a mature primate or rodent PrP2; is raised to a purified human PrP2; is raised to a purified mouse PrP2; is raised against a soluble primate or rodent PrP2^{c}; is raised to a purified soluble human PrP2^{c}; is raised to a purified soluble mouse PrP2^{c}; is raised against an insoluble primate or rodent PrP2^{Sc}; is raised to a purified insoluble human PrP2^{Sc}; is raised to a purified insoluble mouse PrP2^{Sc}; is immunoselected; is a polyclonal antibody; binds to a denatured PrP2; exhibits a Kd to antigen of at least 30 μM; is attached to a solid substrate, including a bead or plastic membrane; is in a sterile composition; or is detectably labeled, including a radioactive or fluorescent label; PrP2 polypeptide, wherein: the polypeptide is a primate or rodent polypeptide; the binding compound is an Fv, Fab, or Fab2 fragment; the binding compound is conjugated to another chemical moiety; or the antibody: is raised against a polypeptide sequence of a mature polypeptide comprising sequence of Table 2; is raised against a mature primate PrP2; is raised to a purified human PrP2; is immunoselected; is a polyclonal antibody; binds to a denatured PrP2; exhibits a Kd to antigen of at least 30 μM; is attached to a solid substrate, including a bead or plastic membrane; is in a sterile composition; or is detectably labeled, including a radioactive or fluorescent label. Kits are provided, e.g., those comprising the binding compound, and: a compartment comprising the binding compound; and/or instructions for use or disposal of reagents in the kit. Preferably, the kit is capable of making a qualitative or quantitative analysis.

Polynucleotide embodiments include an isolated or recombinant polynucleotide encoding a polypeptide or fusion polypeptide, wherein: the PrP2 is from a mammal; said PrP2 nucleic acid: encodes an antigenic peptide sequence of SEQ ID NO: 2, 4, or 6; encodes a plurality of antigenic polypeptide sequences of SEQ ID NO: 2, 4, or 6; exhibits at least about 80% identity to a natural cDNA encoding a PrP2 segment; is in an expression vector; further comprises an origin of replication; is from a natural source; comprises a detectable label; comprises synthetic nucleotide sequence; is less than 6 kb, preferably less than 3 kb; is from a mammal, including a primate; comprises a natural full length coding sequence; is a hybridization probe for a gene encoding said PrP2; comprises a plurality of non-overlapping segments of at least 15, 18, 21, or 25 nucleotides from SEQ ID NO: 1, 3, 5, or 13; or is a PCR primer, PCR product, or mutagenesis primer. The invention further provides a cell comprising such a recombinant PrP2 polynucleotide, e.g., where the cell is: a prokaryotic cell; a eukaryotic cell; a bacterial cell; a yeast cell; an insect cell; a mammalian cell; a mouse cell; a primate cell; or a human cell. Certain kit embodiments include a comprising the polynucleotide, and: a compartment comprising the polynucleotide; a compartment further comprising a primate or rodent PrP2 or primate polypeptide; and/or instructions for use or disposal of reagents in the kit. Preferably, the kit is capable of making a qualitative or quantitative analysis.

In other polynucleotide embodiments, the polynucleotide is one that: hybridizes under wash conditions of 40° C. and less than 2M salt to SEQ ID NO: 3, 5, or 7; exhibits identity over a stretch of at least about 30 nucleotides to a primate or rodent PrP2. In various preferred embodiments: the wash conditions are: at 45° C. and/or 500 mM salt; at 55° C. and/or 150 mM salt; or the stretch is at least 55 nucleotides; or at least 75 nucleotides.

Methods of modulating physiology or development of a cell or tissue culture cells are provided, e.g., comprising contacting the cell with an agonist or antagonist of a primate or rodent PrP2. Preferably, the cell is transformed with a polynucleotide encoding primate PrP2.

### DETAILED DESCRIPTION OF THE PREFERRED EMBODIMENTS

All references herein are incorporated by reference herein in their entirety, as if each individual publication or patent application was specifically and individually indicated to be incorporated by reference; including, without limit, all tables, figures, forms, drawings, and illustrations.

OUTLINE

I. General

II. Activities

III. Nucleic acids

A. encoding fragments, sequence, probes

B. mutations, chimeras, fusions

C. making polynucleotides

D. vectors, cells comprising

IV. Proteins, Peptides

A. fragments, sequence, immunogens, antigens

B. muteins

C. agonists/antagonists, functional equivalents

D. making proteins

V. Making polynucleotides, proteins

A. synthetic

B. recombinant

C. natural sources

VI. Antibodies

A. polyclonals

B. monoclonal

C. fragments; Kd

D. anti-idiotypic antibodies

E. hybridoma cell lines

VII. Kits and Methods to quantify PrP2s

A. ELISA

B. assay mRNA encoding

C. qualitative/quantitative

D. kits

VIII. Therapeutic compositions, methods

A. combination compositions

B. unit dose

C. administration

IX. Binding Compositions

I. General

The present invention provides amino acid and DNA sequences of mammalian, particularly primate and rodent prion-like molecules, designated PrP2, having particular defined properties, both structural and biological. These embodiments increase the number of members of the human prion-like (PrP) family from one to two, but also serve to establish that the family consists of more than a single group of species variants. A cDNA encoding these molecules was obtained from primate, e.g., human; or rodent, e.g., mouse, cDNA sequence libraries. Other primate, rodent, or other mammalian counterparts would also be desired.

Some standard methods applicable are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; or Ausubel, et al. (1987 and periodic supplements) Current Protocols in Molecular Biology, Greene/Wiley, New York; each of which is incorporated herein by reference.

Table 1 shows comparative alignments of the two rodent PrP2s to each other and to primate PrP2. Comparison of rodent and primate PrP2 with other PrP species counterparts is shown in Table 2.

TABLE 1

Alignment and comparison of both rodent PrP2s (respectively,
SEQ ID NOs: 4 & 6). Particularly important segments are:
1-35; 38-127; and 128-176 of SEQ ID NO: 6; and 1-35; 39-128; and
134-179 of SEQ ID NO 4.

| | | | |
|---|---|---|---|
| SEQ ID NO:6 | 1 | MKNRLGTWWVAILCMLLASHLSTVKARGIKHRFKWT-EVLPSGGQITEA | 49 |
| SEQ ID NO:4 | 1 | MKNRLGTWWVAILCMLLASHLSTVKARGIKHRFKWNRKVLPSSGGQITEA | 50 |
| | | ***********************************. ************ | |
| SEQ ID NO:6 | 50 | RVAENRPGAFIKQGRKLDIDFGAEGNRYYAANYWQFPDGIYYEGCSEANV | 99 |
| SEQ ID NO:4 | 51 | RVAENRPGAFIKQGRKLDIDFGAEGNRYYAANYWQFPDGIYYEGCSEANV | 100 |
| SEQ ID NO:6 | 100 | TKEMLVTSCVNATQAANQAEFSREKQD-----RVLWRLIKEICSAKHCDF | 147 |
| SEQ ID NO:4 | 101 | TKEMLVTSCVNATQAANQAEFSREKQDSKLHQRVLWRLIKEICSAKHCDF | 150 |
| | | *************************** ****************** | |
| SEQ ID NO:6 | 148 | WLERGAALRVAVDQPAMVCLLGFVWFIVK | 176 |
| SEQ ID NO:4 | 151 | WLERGAALRVAVDQPAMVCLLGFVWFIVK | 179 |

Alignment and comparison of primate (SEQ ID NO: 2) with rodent PrP2
(SEQ ID NO:6).

| | | | |
|---|---|---|---|
| huPrP2 | 1 | MRKHLSWWWLATVCMLLFSHLSAVQTRGIKHRIKWNRKALPSTAQITEAQ | 50 |
| muPrP2 | 1 | MKNRLGTWWVAILCMLLASHLSTVKARGIKHRFKWTEVLPSSGGQITEAR | 50 |
| | | *. .* **.* .**** ****.*..****** **. * *****. | |
| huPrP2 | 51 | VAENRPGAFIKQGRKLDIDFGAEGNRYYEANYWQFPDGIHYNGCSEANVT | 100 |
| muPrP2 | 51 | VAENRPGAFIKQGRKLDIDFGAEGNRYYAANYWQFPDGIYYEGCSEANVT | 100 |
| | | **************************** ********** * ******** | |
| huPrP2 | 101 | KEAFVTGCINATQAANQGEFQKPDNKLHQQVLWRLVQELCSLKHCEFWLE | 150 |
| muPrP2 | 101 | KEMLVTSCVNATQAANQAEFSREKQDXXXRVLWRLIKEICSAKHCDFWLE | 150 |
| | | ** ** *.******** ** . . .*****..*.** ***.**** | |
| huPrP2 | 151 | RGAGLRVTMHQPVLLCLLALIWLTVK | 176 |
| muPrP2 | 151 | RGAALRVAVDQPAMVCLLGFVWFIVK | 176 |
| | | *** ***.. ** ..*** .* ** | |

Alignment and comparison of primate (SEQ ID NO: 2) with supplemental
rodent PrP2 (SEQ ID NO: 4).

| | | | |
|---|---|---|---|
| huPrP2 | 1 | MRKHLSWWWLATVCMLLFSHLSAVQTRGIKHRIKWNRKALPST-AQITEA | 49 |
| muPrP2 | 1 | MKNRLGTWWVAILCMLLASHLSTVKARGIKHRFKWNRKVLPSSGGQITEA | 50 |
| | | *. .* **.* .**** ****.*..****** ***** ***. ***** | |
| huPrP2 | 50 | QVAENRPGAFIKQGRKLDIDFGAEGNRYYEANYWQFPDGIHYNGCSEANV | 99 |
| muPrP2 | 51 | RVAENRPGAFIKQGRKLDIDFGAEGNRYYAANYWQFPDGIYYEGCSEANV | 100 |
| | | .**************************** ********** * ******* | |
| huPrP2 | 100 | TKEAFVTGCINATQAANQGEFQ--KPDNKLHQQVLWRLVQELCSLKHCEF | 147 |
| muPrP2 | 100 | TKEMLVTSCVNATQAANQAEFSREKQDSKLHQRVLWRLIKEICSAKHCDF | 150 |
| | | *** ** *.******** ** * * ****.*****..*.** ***.* | |
| huPrP2 | 148 | WLERGAGLRVTMHQPVLLCLLALIWLTVK | 176 |
| muPrP2 | 151 | WLERGAALRVAVDQPAMVCLLGFVWFIVK | 179 |
| | | ****** ***.. ** ..*** .* ** | |

TABLE 2

Alignment of PrP2s (SEQ ID NO: 2 and 4) with prion
(PrPs) specie variants (e.g., Bovine PrP SEQ ID NO: 9; Chick PrP
SEQ ID NO: 8; Human PrP SEQ ID NO: 11; Hamster PrP SEQ ID NO: 11;
Mouse PrP SEQ ID NO: 12; and Sheep PrP SEQ ID NO: 7).

| | | |
|---|---|---|
| PrP | BOVIN | ..GG.TH GQWNKPSK.P KTNMKHVAGA AAAGAVVGGL GGYMLGSAMS |
| PrP | CHICK | ..GGSYH NQ..KPWKPP KTNFKHVAGA AAAGAVVGGL GGYAMGRVMS |
| PrP | HUMAN | ..GGGTH SQWNKPSK.P KTNMKHMAGA AAAGAVVGGL GGYMLGSAMS |
| PrP | HAMSTER | ..GGGTH NQWNKPSK.P KTNMKHMAGA AAAGAVVGGL GGYMLGSAMS |
| PrP | MOUSE | ..GGGTH NQWNKPSK.P KTNLKHVAGA AAAGAVVGGL GGYMLGSAMS |
| PrP | SHEEP | ..GG.SH SQWNKPSK.P KTNMKHVAGA AAAGAVVGGL GGYMLGSAMS |
| PrP2 | human | QTRGIKH RI...KWN.. RKALPST.AQ ITEAQVAENR PGAFIKQGR. |
| PrP2 | mouse | KARGIKH RF...KWN.. RKVLPSSGGQ ITEARVAENR PGAFIKQGR. |
| PrP | BOVIN | RPLIHFOSDY EDRYYRENMH RYPNQVYYRP VDQY.SNQNN FVHDCVNITV |
| PrP | CHICK | GMNYHFDSPD EYRWWSENSA RYPNRVYYRD YSSP.VPQDV FVADCFNITV |
| PrP | HUMAN | RPIIHFGSDY EDRYYRENMH RYPNQVYYRP MDEY.SNQNN FVHDCVNITI |
| PrP | HAMSTER | RPMMHFGNDW EDRYYRENMN RYPNQVYYRP VDQY.NNQNN FVHDCVNITI |
| PrP | MOUSE | RPMIHFGNDW EDRYYRENMY RYPNQVYYRP VDQY.SNQNN FVHDCVNITI |
| PrP | SHEEP | RPLIHFGNDY EDRYYRENMY RYPNQVYYRP VDRY.SNQNN FVHDCVNITV |
| PrP2 | HUMAN | KLDIDFGA.E GNRYYEANYW QFPD0IHYNG CSEANVTKEA FVTGCINATQ |
| PrP2 | MOUSE | KLDIDFGA.E GNRYYAANYW QFPDGIYYEG CSEANVTKEM LVTSCVNATQ |
| PrP | BOVIN | KEHTVTTTTK GE........ ..NFTETDIK MMERVVEQMC ITQYQRESQA |
| PrP | CHICK | TEYSIGPAAK KNTSEAVAAA NQTEVEMENK VVTKVIREMC VQQYREYRLA |
| PrP | HUMAN | KQHTVTTTTK GE........ ..NFTETDVK MMERVVEQMC ITQYERESQA |
| PrP | HAMSTER | KQHTVTTTTK GE........ ..NFTETDIK IMERVVEQMC TTQYQKESQA |
| PrP | MOUSE | KQHTVTTTTK GE........ ..NFTETDVK MMERVVEQMC VTQYQKESQA |
| PrP | SHEEP | KQHTVTTTTK GE........ ..NFTETDIK IMERVVEQMC ITQYQRESQA |
| PrP2 | HUMAN | AANQGEFQKP .......... ...DNKLHQQ VLWRLVQELC SLKHCEFWLE |
| PrP2 | MOUSE | AANQAEFSRE KQ........ ...DSKLHQR VLWRLIKEIC SAKHCDFWLE |
| PrP | BOVIN | YYQ..RGASV ILFSSPPVIL LISFLIFLIV G |
| PrP | CHICK | .....SGIQL HPADTWLAVL LLLLTTLFA. . |
| PrP | HUMAN | YYQ..RGSSM VLFSSPPVIL LISFLIFLIV G |
| PrP | HAMSTER | YYDG.RRSSA VLFSSPPVIL LISFLIFLMV G |
| PrP | MOUSE | YYDGRRSSST VLFSSPPVIL LISFLIFLIV G |
| PrP | SHEEP | YYQ..RGASV ILFSSPPVIL LISFLIFLIV G |
| PrP2 | HUMAN | .....RGAGL RVTMHQPVLL CLLALIWLTV K |
| PrP2 | MOUSE | .....RGAAL RVAVDQPAMV CLLGFVWFIV K |

Analysis of primate PrP2 sequence (SEQ ID NO: 2), reveals interesting structural features, e.g., amino residues that define boundaries of segments or fragments of interest under a Parker antigenicity index (Parker, et al. (1986) Biochemistry 19:5425-5432; analyzed using a commercially available program: MacVector™ Ver. 6.5) are segments: 1-22; 22-36; 36-55; 55-63; 63-81; 81-87; 87-104; 104-129; 129-140; 140-148; 148-159; and 159-171; under a Welling antigenicity index (Welling, et al. (1985) FEBS Lett. 188:215-218; MacVector™ Ver. 6.5) segments: 1-9; 9-24; 24-30; 30-38; 38-43; 43-53; 53-62; 62-77; 77-85; 85-95; 95-100; 100-118; 122-135; 137-145; 148-160; and 160-171; and under a protrusion antigenicity index (MacVector™ Ver. 6.5) segments: 1-22; 22-36; 36-80; 85-95; 95-105; 110-128; 128-150; and 158-171.

Further analysis of primate PrP2 sequence (SEQ ID NO: 2), suggests that N-terminal amino residues 1-24 are particularly interesting as defining a predicted signal peptide sequence. See, e.g., the description of signal peptides in The Encyclopedia of Molecular Biology (Sir J. Kendrew ed., 1994). The carboxy residues 154-176 of SEQ ID NO: 2 are also particularly interesting as suggesting that these residues define a GPI (glycosylphosphatidylinositol) anchor-like motif or cell membrane attachment (see, e.g., Brodbeck (1998) Biol. Chem. 379:1041-1044). Recently, it has been demonstrated that glycosylphosphatidylinositol (GPI) anchored proteins possess the potential for cell-cell transfer while remaining both intact and functional. See, e.g., Kooyman, et al., (1998) Exp. Nephrol. 6:148-151. This transfer phenomenon suggests that PrP2 may be transmitted from cell to cell in such a manner. Methods of inhibiting such transfers are encompassed herein as a means to prevent PrP2 prion-like disease transmission. One technique is cleaving and/or releasing PrP2 from its GPI anchor to release it from its associated cell. Another would prevent and/or inhibit such a release from the GPI anchor to maintain the glycosylphosphatidylinositol bond. Potential targets for such methods may include targeting, e.g., hydrolyzing enzymes, such as phospholipase C or D, as they can be associated with a signal mediated release of glycosylphosphatidylinositol anchored proteins either directly by enzymatic cleavage or indirectly, through their effect on the intracellular turnover of glycosylphosphatidylinositols. Furthermore, since, after arrival at the cell membrane, metabolism of the GPI anchor can be effected by diverse means, other inhibitory techniques may also be suitable. See, e.g., Censullo, et al., (1994) Semin. Immunol. 6:81-88. Alternatively, since GPI anchoring requires posttranslational modification in the endoplasmic reticulum via preassembled GPI anchor precursors transferred to proteins bearing a C-terminal GPI signal sequence, it may be possible to selectively modulate the transfer process or manipulate precursor pools to effect GPI expression. See, e.g., Takeda, et al. (1995) Trends Biochem Sci. 20:367-371; and Yeh, et al. (1994) Semin. Immunol. 6:73-80

Particularly interesting segments defining rodent PrP2 (SEQ ID NO: 4) are, e.g., under a Parker antigenicity index (Parker, et al. (1986) Biochemistry 19:5425-5432; analyzed using a commercially available program: MacVector™ Ver. 6.5) segments: 1-20; 20-39; 39-58; 58-64; 64-80; 87-105; 105-132; 132-140; 140-148; 148-152; 152-163; and 165-171; under a Welling antigenicity index (Welling, et al. (1985) FEBS Lett. 188:215-218; MacVector™ Ver. 6.5) segments: 1-20; 20-31; 31-40; 40-54; 54-67; 67-80; 80-112; 112-136; 136-141; and 147-171; and under a protrusion antigenicity index (MacVector™ Ver. 6.5) segments: 1-20; 20-35; 35-51; 56-66; 66-80; 88-95; 95-104; 110-132; 132-140; and 161-171.

Additionally, similar interesting segments define the variant rodent PrP2 (SEQ ID NO: 6). For example, under a Parker antigenicity index (Parker, et al. (1986) Biochemistry 19:5425-5432; analyzed using a commercially available program: MacVector™ Ver. 6.5) segments: 1-21; 21-33; 36-55; 55-63; 63-80; 88-104; 105-127; 127-135; 135-146; 151-160; and 160-171; under a Welling antigenicity index (Welling, et al. (1985) FEBS Lett. 188:215-218; MacVector™ Ver. 6.5) segments: 1-19; 19-32; 32-53; 54-66; 66-82; 82-109; 149-156; and 156-171, under a protrusion antigenicity index (MacVector™ Ver. 6.5) segments: 1-21; 21-33; 33-55; 55-63; 63-79; 79-88; 88-94; 94-103; 110-128; 139-157; and 157-171.

Further analysis of rodent PrP2 sequences (SEQ ID NO: 4 and 6), suggests that the N-terminal amino residues 1-24 are particularly interesting as defining a predicted signal peptide sequence similar to that of SEQ ID NO: 2. Consequently, a mature rodent PrP2 is suggested as amino residues 25-176 for SEQ ID NO: 4 and amino residues 25-173 for SEQ ID NO: 6.

Variant PrP2 (SEQ ID NO: 6) differs from rodent PrP2 (SEQ ID NO: 4) by: (1) the presence of six additional amino acids (R37, S128, K129, L130, H131, and Q132); and (2) the substitution of lysine for glutamic acid at residue 38 (i.e., E38K).

As used herein, the term PrP2 describes a protein or polypeptide comprising a protein or peptide segment having or sharing an amino acid sequence shown in Tables 1 or 2, or antigenic fragment thereof. The invention also encompasses a protein variation of the respective PrP2 alleles whose sequences are provided, e.g., a mutein, or soluble extracellular or intracellular construct. Typically, such agonists or antagonists will exhibit less than about 10% sequence differences, and thus will often have between 1- and 11-fold substitutions, e.g., 2-, 3-, 5-, 7-fold, and others.

It also encompasses allelic, conformational, and other variants, e.g., natural polymorphic, variants of the protein described. The term shall also be used herein to refer to related naturally occurring forms, e.g., alleles, polymorphic variants, species variants, glycoforms, and metabolic variants of the mammalian protein. For example, it has been shown that specific molecular features characterize the protease-resistant prion protein (PrP) detected in animal species as well as in humans infected by the infectious agent strain that causes bovine spongiform encephalopathy (BSE). Studies of glycoform patterns in such diseases in French cattle and cheetahs, as well as in mice infected by isolates from both species, revealed a characteristic molecular signature across PrP variants. Similar studies of 42 French isolates of natural scrapie, from 21 different flocks in different regions of France, however, showed levels of the three glycoforms comparable to those found in BSE-linked diseases. Moreover, the apparent molecular size of the unglycosylated form was also indistinguishable among all different sheep isolates, as well as isolates from BSE in cattle. See, e.g., Baron, et al. (1999) J. Clin. Microbiol. 37:3701-3704.

The invention also encompasses proteins or peptides having substantial amino acid sequence identity with amino acid sequences in Tables 1-2, preferably SEQ ID NO: 2, 4, or 6. It encompasses sequence variants with relatively few substitutions, e.g., typically less than about 25, ordinarily less than about 15, preferably less than about 3-5.

A substantial polypeptide “fragment”, or “segment”, is a stretch of contiguous amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in particularly preferred embodiments are at least 30, 35, 40, 45, 50, 55, 60, 65, 70, 75, 80, 85, 90, 95, 100, 110, 115, 125, and 150 contiguous amino acid residues. Segments comprising sequences of different proteins can be compared one to another over appropriately long stretches of contiguous amino residues. In many cases, the degree of homology or similarity between compared sequences reflects matching a plurality of distinct, e.g., non-overlapping, segments of specified length. Typically, a plurality is at least two, usually at least three, and preferably 5, 7, or more. While length minima are provided, longer lengths and/or combinations of segments with varying lengths is appropriate, e.g., a segment of 7 contiguous residues, and two distinct segments each 12 contiguous residues in length. Similar considerations apply to polynucleotide segments.

Protein or polypeptide homology, or sequence identity, is determined by optimizing residue matches, if necessary, by introducing gaps as required. See, e.g., Needleham, et al. (1970) J. Mol. Biol. 48:443-453; Sankoff, et al. (1983) chapter one in Time Warps, String Edits, and Macromolecules: The Theory and Practice of Sequence Comparison, Addison-Wesley, Reading, Mass.; and software packages from IntelliGenetics, Mountain View, Calif.; and the University of Wisconsin Genetics Computer Group (GCG), Madison, Wis. (each incorporated herein by reference). This changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences are intended to include natural allelic and interspecies variations in the cytokine sequence. Typical homologous proteins or peptides will have from 50-100% homology (if gaps can be introduced), to 60-100% homology (if conservative substitutions are included) with a segment of contiguous amino acids of SEQ ID NO: 2, 4, or 6. Homology measures will be at least about 70%, generally at least 76%, more generally at least 81%, often at least 85%, more often at least 88%, typically at least 90%, more typically at least 92%, usually at least 94%, more usually at least 95%, preferably at least 96%, and more preferably at least 97%, and in particularly preferred embodiments, at least 98% or more. The degree of homology will vary with the length and number of the segments compared. Homologous proteins or peptides, such as the allelic variants, will share most biological activities with the embodiments described in provided in SEQ ID NO: 2, 4, or 6.

As used herein, the term “biological activity” is used to describe, without limitation, PrP2 effects on inflammatory responses, antigenicity, neural activity, circulatory function and disease, innate immunity, and/or morphogenic development, or cell differentiation. For example, the instant PrP2 compositions should mediate disease related activities, activities that are easily assayed using known procedures, e.g., see the assay techniques of U.S. Pat. No. 5,891,641 and references therein which may be adopted for the present PrP2s without requiring undue experimentation. Additional insight into possible PrP2 related disease-states comes from comparing disease associated with PrP specifically and, generally, diseases associated with protein misfolding or protein aggregation. See, e.g., Cohen (1999) J. Mol. Biol. 293:313-320, on the connection between protein folding and neurodegenerative disease and insights into other disorders of protein aggregation and deposition such as Alzheimer's disease.

As the gene structure of PrP2 suggests a secreted protein with a GPI linkage, it is possible that the protein has a soluble stage during its life cycle. As such, it is likely that the protein acts as a soluble messenger having an endocrine-like function with signaling effects via a receptor. Methods of screening for receptors of ligands are well known. See, e.g., Allan et al., (1999) Anal. Biochem. 275:243-247; Kramer et al., (1999) Proteins 37:228-241; Vieira (1998) Mol. Biotechnol. 10:247-250; Cox and Bunce (1999) Anal. Biochem. 267:357-365 Reinscheid et al., (1999) Results Probl. Cell Differ. 26:193-214; Barak et al., (1997) J. Biol. Chem. 272:27497-27500; Stables et al., (1997) Anal. Biochem. 252:115-26; and Mullsand Duggan (1994) Trends Biotechnol 12:47-49 or U.S. Pat. No. 5,989,867.

Moreover, the PrP2 may interact with other proteins, e.g., heterodimer or homodimer forms. Various methodologies exist to identify interacting proteins, e.g., immunoprecipitation, crosslinking, etc. In particular, interaction between a PrP2 and a partner would provide useful insights into the types of interaction in which PrP may participate. Crystallographic analysis of PrP structures would likely parallel those of PrP2, alone or with other partners.

Rational drug design for PrP2s may also be aided based upon structural studies of the molecular shape or conformation of a PrP2, binding compound, or antibody and other effectors or ligands. Effectors of PrP2 may be other proteins that mediate other functions in response to binding with PrP2, or other proteins that normally interact with it. One means for determining which sites interact with specific other proteins is a physical structure determination, e.g., x-ray crystallography or 2 dimensional NMR techniques. These will provide guidance as to which amino residues form contact between PrP2 and other binding partners. For a detailed description of protein structural determination, see, e.g., Blundell and Johnson (1976) Protein Crystallography, Academic Press, New York (incorporated herein by reference). Additional methods of detecting other moieties that bind prion-like compositions are described, e.g., in U.S. Pat. No. 5,679,530. Such techniques can also be applied to PrP2s of the present invention.

II. Activities

The PrP2s will exhibit immunogenic activity, e.g., elicit a selective immune response. Particular techniques to generate antibodies to prion-like compositions can be used with PrP2s of the instant invention. These methods described in U.S. Pat. No. 5,846,533, (incorporated by reference) may be adapted for use with PrP2. Antiserum or antibodies resulting therefrom will exhibit both selectivity and affinity of binding to variable conformational forms of a PrP2 polypeptide, e.g., disease, non-disease, soluble, and non-soluble conformations. Testing for these forms in a biological sample can be carried out, e.g., according to methods adopted for use with PrPs (described in U.S. Pat. No. 5,891,641; incorporated by reference). The PrP2s will also be antigenic, e.g., immunogenic, in binding antibodies raised thereto, in the native, or denatured state. Such will allow immunoprecipitation or immunoselection of selective antibody subsets. Additionally applicable techniques for denaturing or making PrP compositions soluble are described, e.g., in U.S. Pat. No. 5,792,901 (incorporated herein). Antibodies to different conformational or soluble forms of PrP2 can be generated according to methods adopted from U.S. Pat. No. 5,846,533. These PrP methods can also be adapted to PrP2 without requiring undue experimentation.

III. Nucleic Acids

This invention encompasses the use of isolated polynucleotides or fragments thereof, e.g., that encode these or closely related proteins, or fragments thereof, e.g., to encode a corresponding polypeptide, preferably one which is biologically active. In addition, this invention covers isolated or recombinant DNA encoding such proteins or polypeptides of sequences characteristic of the respective PrP2s, either individually or as a group. Typically, the polynucleotide hybridizes, under appropriate conditions, with a polynucleotide coding sequence segment provided in SEQ ID NO: 1, 3, 5, or 13 but preferably not with a corresponding segment of other prion-like compositions. The protein or polypeptide can be a full-length protein or fragment that will typically have a polypeptide segment highly homologous to one shown in SEQ ID NO: 2, 4, or 6. Further, this invention covers the use of isolated or recombinant polynucleotide, or fragments thereof, that encode proteins having fragments equivalent to PrP2s. The isolated polynucleotides can have the respective regulatory sequences in the 5′ and 3′ flanks, e.g., promoters, enhancers, poly-A addition signals, and others from the natural gene.

An “isolated” polynucleotide herein is a polynucleotide, e.g., an RNA, DNA, or a mixed polymer, that is substantially pure, e.g., separated from other components that normally accompany a native sequence, such as ribosomes, polymerases, and flanking genomic sequences from the originating species. The term embraces a nucleic acid sequence removed from its naturally occurring environment, and encompasses recombinant, cloned DNA isolates (distinguishable from naturally occurring compositions) and chemically synthesized analogs or analogs biologically synthesized by heterologous systems. A substantially pure molecule encompasses isolated forms of the molecule, either completely or substantially pure. An isolated polynucleotide will generally be a homogeneous composition of molecules, but, in some embodiments, will exhibit minor heterogeneity due to the presence of some heterologous sequence. This heterogeneity is typically due to heterologous sequence at polymer ends or portions not critical to a desired biological function or activity. A “recombinant” polynucleotide is typically defined by either its method of production or its structure. In reference to its method of production, e.g., a product made by a process, the process may make use of recombinant polynucleotide techniques, e.g., involving human intervention in the nucleotide sequence. Typically, this intervention involves in vitro manipulation, although under certain circumstances it may involve more classical animal breeding techniques. Alternatively, it can be a polynucleotide generated by a sequence comprising fusion of two fragments that are not naturally contiguous, but is meant to exclude products of nature, e.g., naturally occurring mutants as found in their natural state. Thus, for example, products made by transforming cells with an unnaturally occurring vector are encompassed, as are polynucleotides comprising sequence derived using a synthetic oligonucleotide process. Such a process often replaces a codon with a redundant codon encoding the same or a conservative amino acid, while typically introducing or removing a restriction enzyme sequence recognition site. Alternatively, the process joins polynucleotide segments of desired functions to generate a single genetic entity comprising a desired combination of functions not found in the commonly available natural forms, e.g., encoding a fusion protein. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant polypeptide, e.g., fusion polypeptide. This includes dimeric repeats. Specifically included are synthetic polynucleotides that, due to the degeneracy of the genetic code, encode polypeptides equivalent to fragments of PrP2 and fusions of sequences from various different related molecules, e.g., other prion-like members.

A “fragment,” in a polynucleotide context, is a segment of contiguous nucleotides of at least: about 17, generally at least 21, more generally at least 25, ordinarily at least 30, more ordinarily at least 35, often at least 39, more often at least 45, typically at least 50, more typically at least 55, usually at least 60, more usually at least 66, preferably at least 72, more preferably at least 79, and in particularly preferred embodiments will be at least 85 or more contiguous nucleotides, e.g., 100, 120, 140, etc. Typically, fragments of different genetic sequences can be compared to one another over appropriate length stretches, particularly, defined segments such as those described herein. A polynucleotide encoding a PrP2 will be particularly useful to identify genes, mRNA, and cDNA species that code for itself or closely related proteins, as well as DNAs that code for polymorphic, allelic, or other genetic variants, e.g., from different individuals or related species. Preferred probes for such screens are those sequence regions conserved between different polymorphic variants or that contain nucleotides that lack specificity, and will preferably be full length or nearly so, e.g., as shown in Tables 3 or 4. In other situations, polymorphic variant specific sequences will be more useful.

Further, the invention covers recombinant polynucleotide molecules and fragments having polynucleotide sequence identical to or highly homologous to isolated DNA set forth herein. In particular, the sequences will often be operably linked to DNA segments that control transcription, translation, and DNA replication. These additional segments typically assist in expression of a desired polynucleotide segment.

Homologous, or identical polynucleotide sequences, when compared to one another, e.g., PrP2 sequences, exhibit significant similarity. The standards for homology of polynucleotides are measures of homology that are generally based on either sequence matching or on hybridization conditions. Comparative hybridization conditions are described in detail below.

Substantial identity in the polynucleotide sequence comparison context means either that the segments, or their complementary strands, when compared, are identical when optimally aligned, with appropriate nucleotide insertions or deletions, in at least about 60% of the nucleotides, generally at least 66%, ordinarily at least 71%, often at least 76%, more often at least 80%, usually at least 84%, more usually at least 88%, typically at least 91%, more typically at least about 93%, preferably at least about 95%, more preferably at least about 96 to 98% or more, and in particular embodiments, as high at about 99% or more of the nucleotides, including, e.g., segments encoding structural domains such as the segments described below.

Alternatively, substantial identity exists when segments of the compared polynucleotide (typically using a sequence derived from SEQ ID NO: 1, 3, 5, or 13) selectively hybridization to a strand or its complement. Typically, selective hybridization occurs when there is at least about 55% homology over a stretch of at least about 14 nucleotides, more typically at least about 65%, preferably at least about 75%, and more preferably at least about 90%. See, Kanehisa (1984) Nuc. Acids Res. 12:203-213 (incorporated herein by reference). The length of homology comparison, as described, may be over longer stretches, and in certain embodiments is over a stretch of contiguous nucleotides at least about 17, generally at least about 20, ordinarily at least about 24, usually at least about 28, typically at least about 32, more typically at least about 40, preferably at least about 50, and more preferably at least about 75 to 100 or more contiguous nucleotides.

Stringent conditions, in a homology hybridization context, are combinations of salt, temperature, organic solvents, and other parameters typically controlled in hybridization reactions. Stringent temperature conditions here encompass temperatures in excess of about 30° C., more usually in excess of about 37° C., typically in excess of about 45° C., more typically in excess of about 55° C., preferably in excess of about 65° C., and more preferably in excess of about 70° C. Stringent salt conditions encompass less than about 500 mM, usually less than about 400 mM, more usually less than about 300 mm, typically less than about 200 mM, preferably less than about 100 mM, and more preferably less than about 80 mM, even down to less than about 20 mM. However, the combination of parameters is much more important than any single measure. See, e.g., Wetmur and Davidson (1968) J. Mol. Biol. 31:349-370, (incorporated herein by reference). A hybridization signal should be at least 2× over background, generally at least 5-10× over background, and preferably even more.

Typically, for a sequence comparison, one sequence is as a reference to which test sequences are compared. Sequence comparisons may be performed using known algorithms. When using a sequence comparison algorithm, test and reference sequences are input into a computer, subsequent coordinates are designated, if necessary, and sequence algorithm program parameters are designed. An algorithm compares sequences, based on designated program parameters, then calculates the degree of matching (typically given as a percentage) to the reference. Optimal alignments of sequences for comparison are conducted, e.g., by the local homology algorithm of Smith and Waterman (1981) Adv. Appl. Math. 2:482, by the homology alignment algorithm of Needleman and Wunsch (1970) J. Mol. Biol. 48:443, by the similarity search algorithm of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra). An example of a useful comparison algorithm is a method called PILEUP. PILEUP creates a multiple sequence alignment from a group of related sequences using progressive, pairwise alignments to show relationship and percentage identity. It also plots a dendrogram showing the clustering relationships used to create the alignment. PILEUP uses a simplification of the progressive alignment method of Feng and Doolittle (1987) J. Mol. Evol. 35:351-360. The method parallels a method of Higgins and Sharp (1989) CABIOS 5:151-153. The program can align up to 300 sequences, with each sequence having a maximum length of 5,000 nucleotides or amino acids. The multiple alignment procedure begins with the pairwise alignment of the two most similar sequences, producing a cluster of two aligned sequences. This process is iterated so each previous cluster is aligned to the next most related sequence or cluster of aligned sequences. Two clusters of sequences are aligned by a simple extension of the pairwise alignment of two individual sequences. The final alignment is achieved by a series of progressive, pairwise alignments. Program parameters include designating specific sequences and their amino acid or nucleotide coordinates for regions of sequence comparison. For example, a reference sequence can be compared to other test sequences to determine the percent sequence identity relationship using the following parameters: default gap weight (3.00), default gap length weight (0.10), and weighted end gaps.

Another example of an algorithm suitable for comparing sequences is the program BLAST (see, Altschul, et al. (1990) J. Mol. Biol. 215:403-410). Software for performing BLAST is publicly available through the National Center for Biotechnology Information (http:www.ncbi.nlm.nih.gov\). The BLAST algorithm initially identifies high scoring sequence pairs (HSPs) by identifying short words of a designated length (W) in the query sequence that either match or satisfy a positive-valued threshold score (T) when aligned with a word of the same length in a database sequence. The threshold score is referred to as the neighborhood word score threshold (Altschul, et al., supra). These initial neighborhood ‘word’ hits act as seeds for initiating searches to find longer HSPs containing them. The word hits are then extended in both directions along each sequence for as far as the cumulative alignment score can be increased. Extension of word hits are halted when the cumulative alignment score falls off its maximum achieved value by a quantity (X); the cumulative score drops to zero or below (due to the accumulation of one or more negative-scoring residue alignments); or the end of either sequence is reached. The BLAST algorithm parameters W, T, and X determine the sensitivity and speed of the alignment. As default values, BLAST uses a word-length (W) of 11 (the BLOSUM62 scoring matrix), an alignment (B) of 50, an expectation (E) of 10, M=5, and N=4 when comparing both strands (Henikoff and Henikoff (1989) Proc. Nat'l Acad. Sci. USA 89:10915). In addition to calculating percentage sequence identity, BLAST also performs a statistical analysis of the similarity between sequences (see, e.g., Karlin and Altschul (1993) Proc. Nat'l Acad. Sci. USA 90:5873-5787). One measure of sequence similarity used in BLAST is the smallest sum probability (P(N)), which indicates the probability that a match between two sequences is due entirely to chance. For example, a statistically significant, non-random match occurs when the value of the smallest sum probability is less than about 0.1, more preferably less than about 0.01, and most preferably less than about 0.001.

A further indication that two sequences are substantially identical is when, as described below, their respective, encoded polypeptides are immunologically cross-reactive to each other. Thus, by this measure, two polypeptides are typically substantially identical when they differ only by conservative substitutions. Another indicator of substantial identity is the selective hybridization of the two compared sequences under stringent conditions, as described below, to form a detectable nucleotide duplex. The isolated DNA of the invention can be readily modified by nucleotide substitutions, deletions, insertions, and inversions of stretches of contiguous nucleotides. These modifications result in novel DNA sequences encoding a protein of the invention or its derivatives. These modified sequences can be used to produce mutant proteins (muteins) or to enhance the expression of variant species. Enhanced expression can involve gene amplification, increased transcription, increased translation, and other effects. Such mutant PrP2-like derivatives include predetermined or site-specific mutations of the protein or its fragments, including silent mutations using the degeneracy of the genetic code. “Mutant PrP2” as used herein encompasses a polypeptide otherwise falling within the homology definition of PrP2 as set forth herein, but having an amino acid sequence differing from other PrP2s found in nature, whether by way of deletion, substitution, or insertion. In particular, “site specific mutant PrP2” encompasses a protein having substantial homology with a protein of SEQ ID NO: 2, 4, or 6, and typically shares most of the biological activities or effects of the sequences disclosed herein.

Although site-specific mutation sites are predetermined, mutants need not be site specific. Mammalian PrP2 mutagenesis can be achieved by making amino acid insertions or deletions in the gene, coupled with expression. Substitutions, deletions, insertions, or many combinations may be generated to arrive at a final construct. Insertions encompass amino- or carboxy-terminal fusions. Random mutagenesis can be conducted at a target codon and the expressed mammalian PrP2 mutants can then be screened for the desired activity, providing some aspect of a structure-activity relationship. Methods for making substitution mutations at predetermined sites in DNA having a known sequence are well known in the art, e.g., by M13 primer mutagenesis. See also Sambrook, et al. (1989) and Ausubel, et al. (1987 and periodic Supplements) and U.S. Pat. No. 5,679,530.

The mutations in the DNA normally should not place coding sequences out of reading frames and preferably will not create complementary regions that could hybridize to produce secondary mRNA structure such as loops or hairpins.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence.

Polymerase chain reaction (PCR) techniques can often be applied in mutagenesis. Alternatively, mutagenesis primers are commonly used methods for generating defined mutations at predetermined sites. See, e.g., Innis, et al. (eds. 1990) PCR Protocols: A Guide to Methods and Applications Academic Press, San Diego, Calif.; and Dieffenbach and Dveksler (1995; eds.) PCR Primer: A Laboratory Manual Cold Spring Harbor Press, CSH, N.Y. Appropriate primers of length, e.g., 15, 20, 25, or longer can be made using sequence provided.

IV. Proteins, Peptides

As described above, the present invention encompasses primate or rodent PrP2, e.g., sequences disclosed in Tables 1 and 2, and described herein. Allelic and other variants are also contemplated, including, e.g., fusion proteins combining portions of such sequences with others, including epitope tags and functional domains. Particularly interesting constructs include those sequences described above.

The present invention also provides recombinant proteins, e.g., heterologous fusion proteins using segments of PrP2 proteins. A heterologous fusion protein is a fusion of proteins or polypeptide segments that are not normally fused in the same manner. Thus, a fusion product of a PrP2 with another prion or prion-like composition encompasses a continuous polypeptide having sequences fused in a typical peptide linkage, typically made as a single translation product and exhibiting properties, e.g., sequence or antigenicity, derived from each source peptide. A similar concept applies to heterologous polynucleotide sequences.

In addition, new constructs can be made by combining similar functional or structural domains from other related proteins, e.g., PrPs, including PrP2 species variants. For example, binding portions or other segments may be “swapped” creating new fusion polypeptides or fragments. See, e.g., Cunningham, et al. (1989) Science 243:1330-1336; and O'Dowd, et al. (1988) J. Biol. Chem. 263:15985-15992 (each incorporated herein by reference). Thus, chimeric polypeptides, exhibiting new combinations of specificities, will result from the functional linkage of PrP2 binding specificities. For example, domains from related molecules may be added or substituted for domains in this or related proteins. The resulting chimera will often have hybrid function and properties. For example, a fusion protein may include a targeting domain, which serves to sequester the fusion protein to a particular subcellular compartment.

Candidate fusion partners and sequences can be selected from sequence data bases, e.g., GenBank, c/o NCBI, and BCG, University of Wisconsin Biotechnology Computing Group, Madison, Wis. (each incorporated herein by reference).

The present invention also particularly provides PrP2 muteins that are prion-like compositions. Structural alignment of PrP2s with related prion family members illustrate conserved features/residues. See, e.g., Tables 1 and 2.

Similar variations in other species counterparts of prion-like sequences, should provide similar interactions with a ligand or substrate. Substitutions with either rodent or primate sequences, e.g., mouse or human, are particularly preferred.

“Derivatives” of primate or mouse PrP2 include amino acid sequence mutants, glycosylation variants, cleaved variants, conformational variants, metabolic derivatives, solubility variants, and covalent or aggregative conjugates with other chemical moieties. Covalent derivatives can be prepared by linkage of functionalities to groups found in the PrP2 amino acid side chains or at the N- or C- termini, e.g., by common art methods. These derivatives include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanyl aryl species.

In particular, glycosylation alterations are included, e.g., made by modifying the glycosylation patterns of a polypeptide during its synthesis and processing, or in further processing steps. Particularly preferred methods include exposing the polypeptide to glycosylating enzymes derived from cells normally afforded such processing, e.g., mammalian glycosylation enzymes. Also contemplated are deglycosylation enzymes as are versions of the same primary amino acid sequence with other minor modifications, including phosphorylated amino acid residues, e.g., phosphotyrosine, phosphoserine, or phosphothreonine.

Major derivative groups include covalent conjugates of PrP2s, or fragments thereof, with other proteins or polypeptides. These derivatives can be synthesized in recombinant culture such as N- or C-terminal fusions or using other agents in the art known to cross-link proteins through reactive side groups. Preferred derivatization sites with cross-linking agents, include without limit, free amino groups, carbohydrate moieties, and cysteine residues.

Fusion polypeptides between PrP2s and other homologous or heterologous proteins are also provided. Homologous polypeptides encompass any fusions between different prion-like compositions, resulting in, e.g., a hybrid protein with binding specificity for multiple different PrP2s. Likewise, heterologous fusions exhibiting a combination of properties or activities of the derivative proteins can be constructed. Typical examples include fusions of a reporter polypeptide, e.g., luciferase, with a segment or domain of another segment, to indicate the presence or location of a desired PrP2. See, e.g., Dull, et al., U.S. Pat. No. 4,859,609 (incorporated herein by reference). Other gene fusion partners include glutathione-S-transferase (GST), bacterial β-galactosidase, trpE, Protein A, β-lactamase, alpha amylase, alcohol dehydrogenase, and yeast alpha mating factor. See, e.g., Godowski, et al. (1988) Science 241:812-816.

The phosphoramidite method of Beaucage and Carruthers (1981) Tetra. Letts. 22:1859-1862, produces suitable synthetic DNA fragments. A duplex can be formed, under appropriate conditions, either by synthesizing the complementary strand and allowing the strands to anneal or by generating a complementary strand with an appropriate primer sequence and DNA polymerase.

Polypeptides of the invention may also have residues chemically modified by phosphorylation, sulfonation, biotinylation, or by the addition or removal of other moieties, particularly those having molecular shapes similar to phosphate groups. In some embodiments, the modifications will be useful as labeling reagents, or will serve as purification targets, e.g., affinity ligands.

Typically, fusion proteins can be made either by recombinant polynucleotide or by synthetic polypeptide methods. Techniques for polynucleotide manipulation and expression are described generally, e.g., in Sambrook, et al. (1989) Molecular Cloning: A Laboratory Manual (2d ed.), Vols. 1-3, Cold Spring Harbor Laboratory, and Ausubel, et al. (eds. 1987 and periodic supplements) Current Protocols in Molecular Biology, Greene/Wiley, New York, (each incorporated herein by reference). Techniques for polypeptide synthesis are taught, e.g., in Merrifield (1963) J. Amer. Chem. Soc. 85:2149-2156; Merrifield (1986) Science 232: 341-347; and Atherton, et al. (1989) Solid Phase Peptide Synthesis: A Practical Approach, IRL Press, Oxford; all incorporated herein by reference. For methods of making longer polypeptides, see also Dawson, et al. (1994) Science 266:776-779; Muir, et al. (1997) Methods Enzymol. 289:266-298; Sakakibara (1995) Biopolymers 37:17-28; Angeletti, et al. (1997) Methods Enzymol. 289:697-717; Wilken, et al. (1998) Curr. Opin. Biotechnol. 9:412-426; Weiss, et al. (1998) Nat. Struct. Biol. 5:676; Gibney, et al. (1997) Curr. Opin. Chem. Biol. 1:537-542; Hilvert (1994) Chem. Biol. 1:201-203; Hackeng, et al. (1999) Proc. Natl. Acad. Sci. USA. 96:10068-10073; Songster, et al. (1997) Methods Enzymol. 289:126-174; Albericio, et al. (1997) Methods Enzymol. 289:313-336; Camarero, et al. (1998) J. Pept. Res. 51:303-316; and Tam, et al. (1995) Proc. Natl. Acad. Sci. U.S.A. 92(26):12485-12489, each incorporated herein by reference.

The invention also encompasses the use of PrP2 derivatives other than amino acid sequence or glycosylation variants. Such derivatives encompass covalent or aggregative associations with chemical moieties. These derivatives can encompass, for example: (1) salts, (2) side chain and terminal residue covalent modifications, and (3) adsorption complexes, e.g., with cell membranes. Such covalent or aggregative derivatives-are useful as immunogens, as reagents in immunoassays, or in purification methods such as in affinity purification of a binding molecule, e.g., an antibody. For example, a PrP2 can be immobilized by covalent bonding to a solid support such as cyanogen bromide-activated Sepharose, by well known methods in the art (see, e.g., U.S. Pat. No. 5,891,641; incorporated herein), or adsorbed onto polyolefin surfaces, with or without glutaraldehyde cross-linking, for use an assay or to purify a PrP2, antibodies, or other similar molecules. A PrP2 can also be labeled with a detectable group (e.g., radioiodinated by the chloramine T procedure) covalently bound to rare earth chelates, or conjugated to another fluorescent moiety for use in diagnostic assays.

A PrP2 or fragment thereof can be used as an immunogen to produce specific antisera or antibodies, e.g., that distinguish PrP2s, portions thereof, various PrP2 conformations, or other prion family members (PrPs). A purified PrP2 can be used to screen monoclonal antibodies or antigen-binding fragments prepared by immunization with various forms of impure preparations containing PrP2 protein or polypeptide. The term “antibodies” encompasses antigen binding fragments of natural antibodies, e.g., Fab, Fab2, Fv, etc. Purified PrP2s can also be used as reagents to detect antibodies generated to elevated levels of PrP2 expression, or immunological disorders leading to antibody production against endogenous PrP2. Additionally, PrP2 fragments can serve as immunogens to produce antibodies of the invention, as described herein. For example, the invention encompasses antibodies having binding affinity to, or being raised against, polypeptide sequence of SEQ ID NO: 2, 4, or 6, fragments thereof, or homologous peptides. In particular, the invention encompasses antibodies having binding affinity to, or having been raised against, specific fragments that are predicted to be, or actually are, exposed at the exterior protein surface of PrP2 in either disease or non-disease conformations.

Blocking of a physiological response to PrP2 may result, e.g., from inhibiting PrP2 binding to itself or another conformational form, likely through competitive inhibition. Thus, in vitro assays of the present invention will often use antibodies or antigen binding segments of these antibodies, or fragments, attached to solid phase substrates. These assays allow for a diagnostic determination of the effect of either binding region mutations and modifications, or the effect of other mutations and modifications, e.g., those affecting signaling or enzymatic functions.

The invention also encompasses the use of competitive drug screening assays, e.g., using neutralizing antibodies to PrP2, or its fragments, to compete with a test compound for binding to a ligand or other antibody. In this way, neutralizing antibodies or fragments can be used to detect the presence of a polypeptide sharing one or more binding sites of a binding partner and can also be used to occupy binding sites that might otherwise bind a ligand.

V. Making Nucleic Acids and Protein

DNA encoding PrP2 or fragments thereof can be obtained by chemical synthesis, screening cDNA libraries, or by screening genomic libraries prepared from a wide variety of cell lines or tissue samples. Natural sequences can be isolated using standard methods and the sequences provided herein, e.g., in Tables 1 or 2. Other species counterparts can be identified by hybridization techniques, or various PCR methods, combined with searching sequence databases, e.g., GenBank.

The DNAs of the invention can be expressed in a wide variety of host cells to synthesize full-length PrP2 or fragments that can be used, for example, to generate polyclonal or monoclonal antibodies; for binding studies; for construction and expression of modified ligand binding or kinase/phosphatase domains; and for structure/function studies. Variants or PrP2 fragments can be expressed in host cells, which are transformed or transfected with appropriate expression vectors. The expressed molecules can be isolated or purified substantially free of protein or cellular contaminants, other than those derived from the recombinant host, and therefore are particularly useful in pharmaceutical compositions when combined with a pharmaceutically acceptable carrier and/or diluent. The protein, or portions thereof, may be expressed as fusions with other proteins.

Further encompassed herein are expression vectors, typically self-replicating DNA or RNA constructs containing a desired gene or its fragments, usually operably linked to suitable genetic control elements recognized in a suitable host cell. Common art techniques teach which control-elements effect appropriate expression depending on the host cell used. Generally, genetic control elements include a prokaryotic promoter system or a eukaryotic promoter expression control system, a transcriptional promoter, an optional operator to control the onset of transcription, transcription enhancers that elevate mRNA levels, a sequence encoding a suitable ribosome binding site, and sequences that terminate transcription and translation. Expression vectors also usually contain an origin of replication allowing the vector to replicate independently of the host cell.

Vectors encompassed here include those containing polynucleotides encoding PrP2, its fragments, or biologically active equivalent polypeptides. The DNA can be under the control of a viral promoter and can encode a selection marker. Further encompassed here is the use of such vectors to express eukaryotic cDNA coding for a protein of the invention in a prokaryotic or eukaryotic host, where the vector is host compatible and where the eukaryotic cDNA is inserted into the vector such that growth of the recipient host expresses the exogenous cDNA. Usually, expression vectors are designed for stable replication or amplification of the transmitted gene. Replication of the expression vector in the host cell is not necessary, e.g., transient expression of the protein or its fragments is possible using vectors that do not contain a replication origin-recognized by the host. Also encompassed herein, are vectors that integrate the protein or a fragment into the host DNA by recombination.

Vectors encompassed herein without limit, include plasmids, viruses, bacteriophage, integratable DNA fragments, and other vehicles that integrate DNA fragments into a host genome. Specialized vectors, such as expression vectors containing genetic control elements that effect the expression of operably linked genes, are also encompassed. Besides plasmids, all other equivalently functioning vectors are encompassed here. See, e.g., Pouwels, et al. (1985 and Supplements) Cloning Vectors: A Laboratory Manual, Elsevier, N.Y., and Rodriquez, et al. (eds.) Vectors: A Survey of Molecular Cloning Vectors and Their Uses, Buttersworth, Boston, 1988 (both incorporated by reference).

Transformed cells, preferably mammalian cells transformed or transfected with recombinant PrP2 vectors, are encompassed here. Transformed host cells typically express the desired protein or its fragments, but, for purposes of cloning, amplifying, and manipulating its DNA, the transformed cell need not express the exogenous protein. This invention further encompasses culturing transformed cells to produce PrP2. The protein can be recovered, either from the culture or, in certain instances, from the culture medium.

For purposes of this invention, nucleic sequences are operably linked when they are functionally related to each other. For example, DNA for a presequence or secretory leader is operably linked to a polypeptide if it is expressed as a preprotein, if it directs the polypeptide to the cell membrane, or if it participates in the secretion of the polypeptide, e.g., a promoter is operably linked to a coding sequence if it controls the transcription of the polypeptide; or a ribosome-binding site is operably linked to a coding sequence if it is positioned to permit translation. Usually, operably linked, means contiguously attached sequence that is in reading frame, however, certain genetic elements such as repressor genes are not contiguously linked but still bind to operator sequences that can control expression.

Suitable host cells encompassed here include prokaryotes, lower eukaryotes, and higher eukaryotes. Prokaryotes include both gram negative and gram positive organisms, e.g., E. coli, and B. subtilis. Lower eukaryotes include yeasts, e.g., S. cerevisiae and Pichia, and species of the genus Dictyostelium. Higher eukaryotes include established tissue culture cell lines of mammalian and non-mammalian cells, e.g., respectively, human, primate, and rodent cells or insect and avian cells.

Prokaryotic host-vector systems include a variety of vectors for many different species. As used herein, E. coli and its vectors will be used generically to include equivalent vectors used in other prokaryotes. A representative vector for amplifying DNA is pBR322 or many of its derivatives. Vectors that can be used to express the receptor or its fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac (pDR540). See Brosius, et al. (1988) “Expression Vectors Employing Lambda-, trp-, lac-, and Ipp-derived Promoters,” in Vectors: A Survey of Molecular Cloning Vectors and Their Uses, (eds. Rodriguez and Denhardt), Buttersworth, Boston, Chapter 10, pp. 205-236 (each incorporated herein by reference).

Lower eukaryotes, e.g., yeasts and Dictyostelium, may be transformed with PrP2 sequence containing vectors. For purposes of this invention, the most common lower eukaryotic host is baker's yeast (Saccharomyces cerevisiae). It will be used herein to generically represent lower eukaryotes although a number of other strains and species are also available. Of particular interest are the use of two hybrid screens using PrP2 to identify interacting genes that may exist; it may be useful to use different factors that can effect conformational form, e.g., temperature, salt concentration, pH, denaturing agents, etc., as described in U.S. Pat. No. 5,834,593 (incorporated herein). Yeast vectors typically consist of a replication origin (unless of the integrating type), a selection gene, a promoter, DNA encoding the receptor or its fragments, and sequences for translation termination, polyadenylation, and transcription termination. Suitable expression vectors for yeast include such constitutive promoters as 3-phosphoglycerate kinase and various other glycolytic enzyme gene promoters or such inducible promoters as the alcohol dehydrogenase 2 promoter or metallothionine promoter. Suitable vectors include derivatives of the following types: self-replicating low copy number (such as the YRp-series), self-replicating high copy number (such as the YEp-series); integrating types (such as the YIp-series), or mini-chromosomes (such as the YCp-series).

Higher eukaryotic tissue culture cells are normally preferred as host cells for expression of PrP2. In principle, many higher eukaryotic tissue culture cell lines are workable, e.g., insect baculovirus expression systems, whether from an invertebrate or vertebrate source. However, mammalian cells are preferred. Transformation or transfection and propagation of such cells are routine. Examples of useful cell lines include HeLa cells, Chinese hamster ovary (CHO) cell lines, baby rat kidney (BRK) cell lines, insect cell lines, bird cell lines, and monkey (COS) cell lines. Expression vectors for such cell lines usually include an origin of replication, a promoter, a translation initiation site, RNA splice sites (if genomic DNA is used), a polyadenylation site, and a transcription termination site. These vectors also usually contain a selection or amplification gene. Suitable expression vectors encompass plasmids, viruses, or retroviruses carrying promoters derived, e.g., from such sources as from adenovirus, SV40, parvoviruses, vaccinia virus, or cytomegalovirus. Representative examples include pCDNA1; pCD, see Okayama, et al. (1985) Mol. Cell Biol. 5:1136-1142; pMC1neo PolyA, see Thomas, et al. (1987) Cell 51:503-512; and a baculovirus vector such as pAC 373 or pAC 610.

For secreted proteins, an open reading frame usually encodes a polypeptide consisting of a mature or secreted product covalently linked to a signal peptide at its N-terminus. The signal peptide is cleaved before secretion of the mature, or active, polypeptide. The cleavage site can be predicted with a high degree of accuracy using common methods, e.g., von-Heijne (1986) Nucleic Acids Research 14:4683-4690 and Nielsen, et al. (1997) Protein Eng. 10:1-12, and the precise amino acid composition of the signal peptide often does not appear to be critical to its function, e.g., Randall, et al. (1989) Science 243:1156-1159; Kaiser, et al. (1987) Science 235:312-317.

It will often be desired to express these polypeptides in a system that provides a specific or defined glycosylation pattern. In this case, typical glycosylation is provided naturally by the expression system. However, the glycosylation pattern can be modified by exposing the polypeptide, e.g., the unglycosylated form, to appropriate glycosylating proteins introduced into a heterologous expression system. For example, the gene may be co-transformed with one or more genes encoding mammalian or other glycosylating enzymes. Using this approach, certain mammalian glycosylation patterns will be achievable in prokaryote or other cells.

A source of PrP2 can be a eukaryotic or prokaryotic host expressing recombinant PrP2, such as is described above. A source can also be a cell line such as mouse Swiss 3T3 fibroblasts, but other mammalian cell lines are also contemplated by this invention, with the preferred cell line being from the human species.

Now that the sequences are known, the primate PrP2s, fragments, or derivatives thereof can be prepared by conventional processes for synthesizing peptides. These include processes described in Stewart and Young (1984) Solid Phase Peptide Synthesis, Pierce Chemical Co., Rockford, Ill.; Bodanszky and Bodanszky (1984) The Practice of Peptide Synthesis, Springer-Verlag, New York; and Bodanszky (1984) The Principles of Peptide Synthesis, Springer-Verlag, New York (each incorporated herein by reference). For example, an azide process, an acid chloride process, an acid anhydride process, a mixed anhydride process, an active ester process (for example, p-nitrophenyl ester, N-hydroxysuccinimide ester, or cyanomethyl ester), a carbodiimidazole process, an oxidative-reductive process, or a dicyclohexylcarbodiimide (DCCD)/additive process can be used. Solid phase and solution phase syntheses are both applicable to the foregoing processes. Similar techniques can be used with partial PrP2 sequences.

The PrP2 proteins, fragments, or derivatives are suitably prepared in accordance with the above processes as typically employed in peptide synthesis, generally either by a so-called stepwise process that comprises sequentially condensing an amino acid to the terminal amino acid or by coupling peptide fragments to the terminal amino acid. Amino groups that are not being used in the coupling reaction typically must be protected to prevent coupling at an incorrect location.

If a solid phase synthesis is adopted, the C-terminal amino acid is bound to an insoluble carrier or support through its carboxyl group. The insoluble carrier is not particularly limited as long as it has a binding capability to a reactive carboxyl group. Examples of such insoluble carriers include halomethyl resins, such as chloromethyl resin or bromomethyl resin, hydroxymethyl resins, phenol resins, tert-alkyloxy, carbonyl, hydrazidated resins, and the like.

To synthesize the peptide systematically, an amino-group protected amino acid is bound in sequence through condensation of its activated carboxyl group and the reactive amino group of the previously formed peptide or chain. After synthesizing the complete sequence, the peptide is split from the insoluble carrier to produce the peptide. This solid-phase approach is described generally by Merrifield, et al. (1963) in J. Am. Chem. Soc. 85:2149-2156 (incorporated herein by reference).

The prepared protein and fragments thereof can be isolated and purified from the reaction mixture by means of peptide separation, for example, by extraction, precipitation, electrophoresis, various forms of chromatography and the like. The PrP2s of the invention can be obtained in varying degrees of purity depending upon desired uses. Purification can be accomplished using protein purification techniques disclosed herein, see below, or by using antibodies described herein in immunoabsorbant affinity chromatography methods. An immunoabsorbant affinity chromatography is carried out first by linking the antibodies to a solid support and then contacting them with solubilized lysates of appropriate cells, lysates of other cells expressing PrP2, or lysates or supernatants of cells producing PrP2 as a result of DNA techniques, see below.

Generally, purified PrP2 will be at least about 40% pure, ordinarily at least about 50% pure, usually at least about 60% pure, typically at least about 70% pure, more typically at least about 80% pure, preferable at least about 90% pure and more preferably at least about 95% pure, and in particular embodiments, 97%-99% or more. Purity will usually be on a weight basis, but can also be on a molar basis. Different assays may be applied as appropriate.

VI. Antibodies

Antibodies can be raised to the various mammalian PrP2s, e.g., primate PrP2 proteins and fragments thereof, in either naturally occurring, native, recombinant, or disease-related forms. Denatured antigen detection can be useful in, e.g., Western analysis. Anti-idiotypic antibodies useful as agonists or antagonists of a natural PrP2 or an antibody are also encompassed.

Antibodies against predetermined fragments of the protein, including binding fragments and single chain versions, can be raised by immunization of animals with conjugates of the fragments with immunogenic proteins. Monoclonal antibodies are prepared from cells secreting the desired antibody. These antibodies can be screened for binding to normal or defective protein, or screened for agonistic or antagonistic activity. These monoclonal antibodies will usually bind with at least a K_{D} of about 1 mM, more usually at least about 300 μM, typically at least about 100 μM, more typically at least about 30 μM, preferably at least about 10 μM, and more preferably at least about 3 μM or better.

The antibodies, including antigen-binding fragments, of the invention can have significant diagnostic or therapeutic value. They can be potent antagonists by binding and inhibiting other compositions from binding; or they can inhibit the ability of a prion-like composition to elicit a biological response, e.g., act on its substrate. They can also be useful as non-neutralizing antibodies and can be coupled to toxins or radionuclides to bind producing cells, or cells localized to the source of the prion. Further, these antibodies can be conjugated to drugs or other therapeutic agents, either directly or indirectly by means of a linker.

The antibodies of the invention are also useful in diagnostic applications, e.g., as capture or non-neutralizing antibodies, they can bind without inhibiting an appropriate substrate binding. As neutralizing antibodies, they are useful in competitive binding assays. They are also useful to detect or quantify PrP2 such as, Western blot analysis, or for immunoprecipitation or immunopurification of the respective protein.

PrP2 fragments can be joined to other materials, particularly polypeptides, as fused or covalently joined polypeptides for use as immunogens. Mammalian PrP2s and fragments can be fused or covalently linked to a variety of immunogens, such as keyhole limpet hemocyanin, bovine serum albumin, tetanus toxoid, etc. See Microbiology, Hoeber Medical Division, Harper and Row, 1969; Landsteiner (1962) Specificity of Serological Reactions, Dover Publications, New York; and Williams, et al. (1967) Methods in Immunology and Immunochemistry, Vol. 1, Academic Press, New York; each incorporated herein by reference, for descriptions of methods of preparing polyclonal antisera. A typical method involves hyperimmunization of an animal with an antigen. The blood of the animal is then collected shortly after the repeated immunizations and the gamma globulin is isolated.

In some instances, it is desirable to prepare monoclonal antibodies from various mammalian hosts, such as mice, rodents, primates, humans, etc. Description of techniques for preparing such monoclonal antibodies can be found in, e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.) Academic Press, New York; and particularly in Kohler and Milstein (1975) in Nature 256:495-497, discussing a method of generating monoclonal antibodies. Summarized briefly, an animal is injected with an immunogen, sacrificed, then spleen cells are removed and fused with myeloma cells to produce a “hybridoma” that can reproduce in vitro. A population of hybridomas is generated, then screened to isolate individual clones, each of which secrete a single antibody that is species specific to the immunogen. Every antibody specie obtained by this method is generated in response to a specific site recognized on the immunogenic substance.

Other suitable techniques encompassed here include in vitro exposure of lymphocytes to the antigenic polypeptides or, alternatively, to selection of libraries of antibodies in phage or similar vectors. See, Huse, et al. (1989) “Generation of a Large Combinatorial Library of the Immunoglobulin Repertoire in Phage Lambda,” Science 246:1275-1281; and Ward, et al. (1989) Nature 341:544-546 (each incorporated herein by reference). The polypeptides and antibodies of the present invention can be used with or without modification, including chimeric or humanized antibodies. Frequently, the polypeptides and antibodies will be labeled by joining, either covalently or non-covalently, a substance that gives a detectable signal. A variety of such labels and conjugation techniques are commonly known. Suitable labels include, but are not limited to, radionuclides, enzymes, substrates, cofactors, inhibitors, fluorescent moieties, chemiluminescent moieties, magnetic particles, and the like. Methods teaching the use of such labels include U.S. Pat. Nos. 3,817,837; 3,850,752; 3,939,350; 3,996,345; 4,277,437; 4,275,149; and 4,366,241. Recombinant or chimeric immunoglobulins may also be produced by the method of U.S. Pat. No. 4,816,567 or in transgenic mice (see, Mendez, et al. (1997) Nature Genetics 15:146-156; both are incorporated herein by reference).

Antibodies of the invention can also be used to isolate PrP2 by affinity chromatography. Columns are prepared linking antibodies to a solid support, e.g., particles, such as agarose, Sephadex, or the like, where a cell lysate is passed through the column, followed by a column wash then increasing concentrations of a mild denaturant to release purified protein for subsequent use in purifying antibody.

The antibodies can also be used to screen expression libraries for particular expression products. Typically, such procedures use labeled antibodies to detect the presence of antigen:antibody complex.

Antibodies raised against PrP2 can be used to produce anti-idiotypic antibodies. These antibodies are useful to detect or diagnose various immunological conditions related to PrP2 expression. They can also be used as agonists or antagonists of a composition that competitively inhibits or substitutes for a naturally occurring ligand.

A PrP2 protein that specifically binds to, or is specifically immunoreactive with, an antibody generated against a defined immunogen (such as an immunogen consisting of the amino acid sequence of SEQ ID NO: 2, 4, or 6) is typically determined in an immunoassay. The immunoassay typically uses a polyclonal antiserum raised to a protein, e.g., of SEQ ID NO: 2, 4, or 6. Such antiserum is selected for low crossreactivity to other prion family members, e.g., PrPs, preferably from the same species, and any crossreactivity is removed by immunoabsorption before use of the antiserum in an immunoassay.

To produce antisera for immunoassay use, a PrP2 (e.g., SEQ ID NO: 2, 4, or 6) is isolated e.g., as described in U.S. Pat. Nos.: 5,891,641, 5,834,593, 5,846,533, and 5,792,901 (each incorporated herein by reference). Additionally, recombinant protein can be produced in a mammalian cell line. An appropriate host, e.g., an inbred strain of mice such as Balb/c, is immunized with a selected protein, typically using a standard adjuvant, such as Freund's adjuvant, and a standard mouse immunization protocol (see Harlow and Lane, supra). Alternatively, a synthetic peptide, derived from the sequences disclosed herein and conjugated to a carrier protein, can be used an immunogen. Polyclonal sera are collected and titered against the immunogen protein in an immunoassay, e.g., a solid phase immunoassay with the immunogen immobilized on a solid support. Polyclonal antisera with a titer of 10⁴ or greater are selected and tested for cross reactivity to other prion family members using a competitive binding immunoassay (such as described supra, Harlow and Lane pp. 570-573). Preferably, at least two PrP2s or PrP family members are used in this determination. These prion family members can be produced as recombinant proteins and isolated using standard molecular biology and protein chemistry techniques.

Competitive binding immunoassays can be used to determine crossreactivity. For example, a mature polypeptide of SEQ ID NO: 2, 4, or 6 is immobilized to a solid support, then proteins are added to competitively bind for the immobilized antigen. The ability of the proteins to compete with the antisera to bind an immobilized antigen is compared to other prions, e.g., PrP2 or PrPs. A degree of crossreactivity is then calculated. Antisera with less than 10% crossreactivity to each test protein are selected and pooled. Cross-reacting antibodies are then removed from pooled antisera by immunoabsorption using the above-listed proteins.

The immunoabsorbed and pooled antisera are subsequently used in a competitive binding immunoassay to compare a second protein to the immunogen protein (e.g., a PrP2 polypeptide of SEQ ID NO: 2, 4, or 6). To make this comparison, the two proteins are each assayed at a wide range of concentrations. For each protein, an amount is determined indicating that the ability of antisera to bind the immobilized protein has been reduced by 50%. If the amount required to inhibit binding for the second protein is less than twice the amount required for the selected test protein or proteins, then the second protein is said to specifically bind to an antibody generated to the immunogen.

It is to be understood that PrP2s are members of a family of homologous proteins. For a particular gene product, such as PrP2, the term refers not only to the amino acid sequences disclosed herein, but also to other allelic, non-allelic, or species variants. Also encompassed by the term PrP2 are non-natural deliberate mutations by conventional recombinant technology such as by single site mutation, excising short sections of DNA encoding the respective proteins, substituting new amino acids, or by adding new amino acids. Typically, such alterations substantially maintain the immunoidentity of the original molecule and/or its biological activity. Thus, these alterations encompass proteins specifically immunoreactive with a designated naturally occurring PrP2 protein. The biological properties of the altered proteins are determined by expressing the protein in an appropriate cell line and measuring the appropriate effect, e.g., upon transfected lymphocytes. Particular minor protein modifications encompassed here include conservative substitutions of amino acids with similar chemical properties, as described above for the PrP2 family as a whole. By optimally aligning a protein with PrP2s and by using conventional immunoassays to determine immunoidentity, one can determine the protein compositions of the invention.

VII. Kits and quantitation

Both naturally occurring and recombinant PrP2 forms are particularly useful in kits and assay methods. For example, these methods can be applied to screening for PrP2 binding activity, e.g., ligands, or binding compositions of PrP2s. Methods to automate such assays have recently been developed to screen tens of thousands of compounds. See, e.g., a BIOMEK automated workstation, Beckman Instruments, Palo Alto, Calif., and Fodor, et al. (1991) Science 251:767-773, (incorporated herein by reference). The latter reference teaches methods of screening for potential binding compositions by synthesizing a plurality of defined polymers on a solid substrate. The development of suitable assays to screen for a ligand or agonist/antagonist homologous proteins is greatly facilitated by the availability of large amounts of purified, isolated, soluble, or insoluble PrP2. Techniques for generating soluble PrP2s can be adapted, without undue experimentation, from, e.g., U.S. Pat. No. 5,843,593 (hereby incorporated by reference) describing methods of preparing soluble PrP.

Purified PrP2 can be coated directly onto plates for use in the above screening assays. However, non-neutralizing antibodies to these proteins can be used as capture antibodies to immobilize a PrP2 on the solid phase, e.g., in diagnostic uses.

The invention also encompasses the use of PrP2, or fragments thereof, peptides, and PrP2 fusion products in a variety of diagnostic kits and methods for detecting the presence of the protein in a sample, such as, e.g., described in U.S. Pat. No. 5,908,969 (hereby incorporated by reference). Alternatively, or additionally, antibodies against molecules of the invention may be incorporated into the kits and methods. Typically, the kit has a compartment/s containing either a PrP2 peptide or gene segment or reagent recognizing one or the other. Typically, recognition reagents, in the case of peptide, would be a binding compound or antibody, or in the case of a gene segment, would usually be a hybridization probe.

A preferred kit for determining the concentration of PrP2 in a sample typically comprises a labeled compound, e.g., ligand or antibody, having known binding affinity for PrP2, a source of PrP2 (naturally occurring or recombinant) as a positive control, and a means for separating bound from free-labeled compound, for example a solid phase for immobilizing the PrP2 of the test sample. Compartments containing reagents, and instructions, will normally be provided.

Antibodies specific for mammalian PrP2, including antigen binding fragments or a peptide fragment/s, are useful in diagnostic applications to detect elevated levels of PrP2 and/or its fragments. Diagnostic assays may be homogeneous (without a separation step between free reagent and antibody-antigen complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA) and the like. For example, unlabeled antibodies can be employed by using a labeled second antibody that recognizes an antibody to PrP2 or to a particular fragment thereof. Such assays are well known in the art, see, e.g., Harlow and Lane (1988) Antibodies: A Laboratory Manual, CSH., and Coligan (ed. 1991) and periodic supplements, Current Protocols In Immunology Greene/Wiley, New York.

Anti-idiotypic antibodies may have similar use to serve as agonists or antagonists of PrP2s. Under appropriate circumstances, these should be useful as therapeutic reagents.

Frequently, to optimize assay sensitivity, reagents for diagnostic assays are supplied in kits. For the subject invention, depending upon the nature of the assay, the protocol, and the label, either labeled or unlabeled antibody, or labeled ligand is provided. This is usually in conjunction with other additives, such as buffers, stabilizers, materials necessary for signal production such as substrates for enzymes, and the like. Typically the kit has compartments for each useful reagent, and will contain instructions for proper use and disposal of reagents. Preferably, reagents for performing an assay are provided as a dry lyophilized powder that can be reconstituted, to appropriate concentrations, in an aqueous medium.

The aforementioned diagnostic constituents can be used with or without modification, for example, labeling can be achieved by joining a moiety (covalently or non-covalently), which provides a detectable signal (directly or indirectly). In many of these assays, a test compound, PrP2, or antibodies thereto is labeled either directly or indirectly. Direct labels include, e.g., radiolabels such as ¹²⁵I, enzymes such as peroxidase and alkaline phosphatase (U.S. Pat. No. 3,645,090), and fluorescent labels (U.S. Pat. No. 3,940,475) capable of monitoring the change in fluorescence intensity, wavelength shift, or fluorescence polarization. Types of indirect labeling encompassed include biotinylation of a constituent followed by binding a labeled avidin moiety.

Numerous methods are known to separate bound from free ligand or bound from free test compound. A PrP2 is immobilized on a matrix followed by washing. Suitable matrices include, without limit, plastics such as in ELISA plates, filters, and beads. Methods of matrix immobilization include, without limit, direct adhesion to plastic, use of a capture antibody, chemical coupling, and biotin-avidin. The last step in such an approach involves precipitation of an antibody:antigen complex by any method including using, e.g., an organic solvent such as polyethylene glycol or a salt such as ammonium sulfate. Other suitable separation techniques include, without limitation, the fluorescein antibody magnetizable particle method of Rattle, et al. (1984) Clin. Chem. 30(9):1457-1461, and the double antibody magnetic particle separation as described in U.S. Pat. No. 4,659,678 (each incorporated herein by reference).

Methods for linking protein or fragments to various labels are well known in the art. Many involve the use of activated carboxyl groups either by using carbodiimide or active esters to form peptide bonds, the formation of thioethers by reaction of a mercapto group with an activated halogen such as chloroacetyl, or an activated olefin such as maleimide, for linkage, or the like. Fusion proteins are also useful in these applications.

Another diagnostic aspect of the invention uses oligonucleotide or polynucleotide sequences of a PrP2. These sequences can be used as probes to detect PrP2 in patients suspected of having a prion disorder. Preparation of both RNA and DNA nucleotide sequences, labeling of the sequences, and determining their preferred size of the sequences are all well known in the art. Typically, an oligonucleotide probe should have at least about 14 nucleotides, usually at least about 18 nucleotides, and the polynucleotide probes may be up to several kilobases. Various labels can be employed, most commonly radionuclides, particularly ³²p. However, other techniques can also be used, such as incorporating biotin-modified nucleotides into a polynucleotide. The biotin serves as a binding site for avidin or antibodies, which can also be labeled. Alternatively, antibodies can be used that recognize specific duplex formations, including DNA:DNA duplexes, RNA:RNA duplexes, DNA:RNA hybrid duplexes, or DNA:protein duplexes. Antibodies can be labeled and an assay carried out where a duplex is bound to a surface, so that upon formation, the bound antibody:duplex is detectable. Probing novel anti-sense RNAs can be carried out using conventional techniques such as polynucleotide hybridization, plus and minus screening, recombinational probing, hybrid released translation (HRT), and hybrid arrested translation (HART). “Such techniques encompasses without limit any amplification using polymerase chain reaction (PCR).

Diagnostic kits that also test for qualitative or quantitative markers are also encompassed here. Diagnosis or prognosis may rely on a combination of markers used as multiple indicators. Thus, kits encompassed here, include testing for combinations of markers. See, e.g., Viallet, et al. (1989) Progress in Growth Factor Res. 1:89-97.

VIII. Therapeutic Utility

The invention provides reagents having significant therapeutic value, such as, PrP2s (naturally occurring or recombinant), fragments thereof, mutein receptors, and antibodies, along with compounds identified as having binding affinity to the PrP2s or antibodies. These reagents are useful in ameliorating conditions of abnormal prion expression. Some prion-like conditions require participation of immune system cells. See, Scrapie replication in lymphoid tissues depends on prion protein-expressing follicular dendritic cells by Brown, et al. (1999) Nature Medicine 5: 1308-12. Additionally, the invention provides therapeutic value for various conditions or states associated with abnormal expression or abnormal triggering of response to a prion, or prion-like composition. See, e.g., Prusiner (1991) Science 252:1515-1522; Wilesmith and Wells (1991) Microbiol. Immunol. 172:21-38; Gajdusek (1977) Science 197:943-960; and Medori, et al. (1992) N. Engl. J. Med. 326:444-449.

Recombinant PrP2s, muteins, agonist or antagonist antibodies thereto, or PrP2 antibodies can be purified and then administered to ameliorate a patient's condition. These reagents can be combined for therapeutic use with additional active ingredients, e.g., in conventional pharmaceutically acceptable carriers or diluents, along with physiologically innocuous stabilizers and excipients. Such combinations can be made sterile (e.g., filtered) and placed into appropriate dosage forms (e.g., by lyophilization in vials or by storage in stabilized aqueous preparations). The invention further encompasses the use of antibodies or binding fragments thereof, which do not bind complement.

The quantity of reagent to effect therapy depends on many factors, including means of administration, target site, physiological half-life, pharmacological shelf-life, physiological state of the patient, and other administered medicants. Thus, treatment dosage is titrated to optimize safety and efficacy. Typically, in vitro dosage may guide in situ dosage. Furthermore, animal testing will circumscribe dosage ranges useful for humans. Various such considerations are described, e.g., in Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa. (each incorporated herein by reference). Methods of administration e.g., oral, intravenous, intraperitoneal, intramuscular, transdermal diffusion, and others, are discussed therein and below. Pharmaceutically acceptable carriers include, without limit, water, saline, buffers, and other compounds described, e.g., in the Merck Index, Merck & Co., Rahway, N.J. Because of the likelihood of high affinity binding or high turnover, low dosages of these reagents would initially be expected as effective. Thus, dosage ranges, including an appropriate carrier, are ordinarily expected to be in concentrations lower than 1 mM, typically less than about 10 μM, usually less than about 100 nM, preferably less than about 10 pM (picomolar), and most preferably less than about 1 fM (femtomolar). Slow release formulations or a slow release apparatus can be utilized for continuous administration.

The PrP2s, fragments thereof, and PrP2 antibodies or its fragments, antagonists, and agonists, may be administered directly to the host to be treated or, depending on the size of the compounds, may, prior to administration, be conjugated to carrier proteins such as ovalbumin or serum albumin. Therapeutic formulations may be administered in many conventional dosage formulations. While the active ingredient may be administered in isolation, it is preferably presented in a pharmaceutical formulation. As defined above, such formulations comprise at least one active ingredient together with an acceptable carrier/s. Each carrier must be both pharmaceutically and physiologically compatible with all other ingredients and safe. Typically, formulations encompassed herein, without limit, are those suitable for oral, rectal, nasal, or parenteral (including subcutaneous, intramuscular, intravenous, and intradermal) administration. A formulation may be conveniently presented in unit dosage form and by methods well known in the pharmaceutical arts. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al. (eds. 1990) Pharmaceutical Dosage Forms: Disperse Systems Dekker, NY (all incorporated by reference). A therapeutic formulation of this invention may be combined or used in with other therapeutic agents, e.g., with agonists or antagonists of other prion-like compositions.

IX. Binding Compositions

To identify molecules having binding affinity to the PrP2s, screening can be performed using PrP2 or fragments thereof. Subsequently, biological assays can be used to determine if a putative binding composition competitively binds and blocks intrinsic stimulating activity, e.g., seeding conformational change of a normal PrP2 to an abnormal or insoluble form. PrP2s fragments can be used to block or antagonize the activity of a PrP2 ligand, or to interfere with PrP2 binding to a binding partner, e.g., either homo- or heterodimeric binding or a receptor-like binding of PrP2. Likewise, a composition having intrinsic stimulating activity can activate or agonize, e.g., by inducing signaling. The invention further encompasses the use of PrP2 antibodies as therapeutic antagonists or agonists similar to the uses proposed for PrP agonists or antagonists in, e.g., U.S. Pat. No: 5,846,533 (incorporated herein).

The PrP2s herein provide means to identify PrP2 binding compounds. Such a ligand or binding compound should specifically bind to a respective PrP2 with reasonably high affinity. Typically, binding constants will be at least about 30 mM, e.g., generally at least about 3 mM, more generally at least about 300 μM, typically at least about 30 μM, 3 μM, 300 nM, 30 nM, etc. Various constructs encompassed herein allow labeling of a PrP2 to detect its ligand. For example, directly labeling a PrP2, fusing markers onto it for secondary labeling, e.g., FLAG or other epitope tags, etc., will allow detection, such as histological detection, as an affinity method for biochemical purification, or by detection in an expression cloning approach. For example, a two-hybrid selection system can also be applied making appropriate constructs with available PrP2 sequences, e.g., see, Fields and Song (1989) Nature 340:245-246.

Generally, PrP2 descriptions are applicable by analogy to specific individual embodiments directed to PrP2 and/or reagents and compositions.

The broad scope of this invention is best understood with reference to the following specific examples, however, these examples are not intended to limit, in any manner, the invention to these embodiments.

### EXAMPLES

I. General Methods

Some standard methods useful herein are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al. Biology Greene Publishing Associates, Brooklyn, N.Y.; or Ausubel, et al. (1987 and Supplements) Current Protocols in Molecular Biology, Greene/Wiley, New York. Methods for protein purification encompass such methods as ammonium sulfate precipitation, column chromatography, electrophoresis, centrifugation, crystallization, and others. See, e.g., Ausubel, et al. (1987 and periodic supplements); Coligan, et al. (ed. 1996 and periodic supplements) Current Protocols In Protein Science Greene/Wiley, New York; Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combinations using recombinant techniques allow fusion to appropriate segments, e.g., to a FLAG sequence or an equivalent that can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System QUIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank, NCBI, SWISSPROT, and others.

Many techniques applicable to other prion compositions (e.g., PrPs) as described, e.g., in U.S. Pat. Nos.: 5,891,641, 5,834,593, 5,846,533, 5,792,901, 5,679,530, and 5,908,969 (all of which are incorporated herein by reference for all purposes) can be applied to PrP2s of the present invention. While many of the techniques and reagents described therein are directed to PrP, corresponding methods and reagents are typically applicable to PrP2s without necessitating undue experimentation.

II. Computational Analysis.

Human PrP2 sequences related to published prion sequences (PrPs) were identified from various EST databases using, e.g., the BLAST server. Altschul, et al. (1994) Nature Genet. 6:119-129. More sensitive pattern- and profile-based methods identified gene fragments exhibiting identity to previously described PrP genes, see, e.g., Bork and Gibson (1996) Meth. Enzymol. 266:162-184.

III. Cloning of full-length human prion cDNAs.

PCR primers derived from the PrP2 sequences are used to probe an appropriate human cDNA library to yield a full length PrP2 or cDNA sequence, see, e.g., Nomura, et al. (1994) DNA Res. 1:27-35. Full-length cDNAs for human PrP2 are cloned, e.g., by common DNA hybridization screening techniques. PCR reactions are conducted using T. aquaticus Taqplus DNA polymerase (Stratagene) under appropriate conditions.

IV. Localization of PrP2 mRNA

Human multiple tissue (Cat. No. 1, or 2) and cancer cell line blots (Cat. No. 7757-1), containing approximately 2 μg of poly(A)⁺ RNA per lane, are purchased from Clontech (Palo Alto, Calif.). Probes are radiolabeled with [γ³²P] dATP, e.g., using the Amersham Rediprime random primer labeling kit (RPN1633). Prehybridization and hybridizations are performed at 65° C. in 0.5 M Na₂HPO₄, 7% SDS, 0.5 M EDTA (pH 8.0). High stringency washes are conducted, e.g., at 65° C. with two initial washes in 2×SSC, 0.1% SDS for 40 min followed by a subsequent wash in 0.1×SSC, 0.1% SDS for 20 min. Membranes are then exposed at −70° C. to x-ray film (Kodak) in the presence of intensifying screens. More detailed studies are performed using cDNA library Southerns with selected human PrP2 clones to examine their expression in hemopoietic or other cell subsets.

Two prediction algorithms that take advantage of the patterns of conservation and variation in multiply aligned sequences, PHD (Rost and Sander (1994) Proteins 19:55-72) and DSC (King and Sternberg (1996) Protein Sci. 5:2298-2310) are used.

Alternatively, two appropriate primers are created using sequence selected from Tables 1 or 2. RT-PCR is used on an appropriate mRNA sample selected for the presence of message to produce a cDNA, e.g., a sample that expresses the gene.

Full-length clones may be isolated by hybridization of cDNA libraries from appropriate tissues pre-selected by PCR signal. Northern blots can be performed.

Message for genes encoding, e.g., PrP2 will be assayed by appropriate technology, e.g., PCR, immunoassay, hybridization, or otherwise. Tissue and organ cDNA preparations are available, e.g., from Clontech, Mountain View, Calif. Identification of sources of natural expression is useful, as described.

Southern Analysis on cDNA libraries can be performed: DNA (5 μg) from a primary amplified cDNA library is digested with appropriate restriction enzymes to release the inserts, run on a 1% agarose gel and transferred to a nylon membrane (Schleicher and Schuell, Keene, N.H.).

Samples for human mRNA isolation may include, e.g.: peripheral blood mononuclear cells (monocytes, T cells, NK cells, granulocytes, B cells), resting (T100); peripheral blood mononuclear cells, activated with anti-CD3 for 2, 6, 12 h pooled (T101); T cell, TH0 clone Mot 72, resting (T102); T cell, THO clone Mot 72, activated with anti-CD28 and anti-CD3 for 3, 6, 12 h pooled (T103); T cell, TH0 clone Mot 72, anergic treated with specific peptide for 2, 7, 12 h pooled (T104); T cell, TH1 clone HY06, resting (T107); T cell, TH1 clone HY06, activated with anti-CD28 and anti-CD3 for 3, 6, 12 h pooled (T108); T cell, TH1 clone HY06, anergic treated with specific peptide for 2, 6, 12 h pooled (T109); T cell, TH2 clone HY935, resting (T110); T cell, TH2 clone HY935, activated with anti-CD28 and anti-CD3 for 2, 7, 12 h pooled (T111); T cells CD4+CD45RO— T cells polarized 27 days in anti-CD28, IL-4, and anti IFN-γ, TH2 polarized, activated with anti-CD3 and anti-CD28 4 h (T116); T cell tumor lines Jurkat and Hut78, resting (T117); T cell clones, pooled AD130.2, Tc783.12, Tc783.13, Tc783.58, Tc782.69, resting (T118); T cell random γβ T cell clones, resting (T119); Splenocytes, resting (B100); Splenocytes, activated with anti-CD40 and IL-4 (B101); B cell EBV lines pooled WT49, RSB, JY, CVIR, 721.221, RM3, HSY, resting (B102); B cell line JY, activated with PMA and ionomycin for 1, 6 h pooled (B103); NK 20 clones pooled, resting (K100); NK 20 clones pooled, activated with PMA and ionomycin for 6 h (K101); NKL clone, derived from peripheral blood of LGL leukemia patient, IL-2 treated (K106); NK cytotoxic clone 640-A30-1, resting (K107); hematopoietic precursor line TF1, activated with PMA and ionomycin for 1, 6 h pooled (C110); U937 premonocytic line, resting (M100); U937 premonocytic line, activated with PMA and ionomycin for 1, 6 h pooled (M101); elutriated monocytes, activated with LPS, IFNγ, anti-IL-10 for 1, 2, 6, 12, 24 h pooled (M102); elutriated monocytes, activated with LPS, IFNγ, IL-10 for 1, 2, 6, 12, 24 h pooled (M103); elutriated monocytes, activated with LPS, IFNγ, anti-IL-10 for 4, 16 h pooled (M106); elutriated monocytes, activated with LPS, IFNγ, IL-10 for 4, 16 h pooled (M107); elutriated monocytes, activated LPS for 1 h (M108); elutriated monocytes, activated LPS for 6 h (M109); DC 70% CD1a+, from CD34+ GM-CSF, TNFβ 12 days, resting (D101); DC 70% CD1a+, from CD34+ GM-CSF, TNFβ 12 days, activated with PMA and ionomycin for 1 hr (D102); DC 70% CD1a+, from CD34+ GM-CSF, TNFβ 12 days, activated with PMA and ionomycin for 6 hr (D103); DC 95% CD1a+, from CD34+ GM-CSF, TNFβ 12 days FACS sorted, activated with PMA and ionomycin for 1, 6 h pooled (D104); DC 95% CD14+, ex CD34+ GM-CSF, TNFβ 12 days FACS sorted, activated with PMA and ionomycin 1, 6 hr pooled (D105); DC CD1a+ CD86+, from CD34+ GM-CSF, TNFβ 12 days FACS sorted, activated with PMA and ionomycin for 1, 6 h pooled (D106); DC from monocytes GM-CSF, IL-4 5 days, resting (D107); DC from monocytes GM-CSF, IL-4 5 days, resting (D108); DC from monocytes GM-CSF, IL-4 5 days, activated LPS 4, 16 h pooled (D109); DC from monocytes GM-CSF, IL-4 5 days, activated TNFβ, monocyte supernatant for 4, 16 h pooled (D110); leiomyoma L11 benign tumor (X101); normal myometrium M5 (O115); malignant leiomyosarcoma GS1 (X103); lung fibroblast sarcoma line MRC5, activated with PMA and ionomycin for 1, 6 h pooled (C101); kidney epithelial carcinoma cell line CHA, activated with PMA and ionomycin for 1, 6 h pooled (C102); kidney fetal 28 wk male (O100); lung fetal 28 wk male (O101); liver fetal 28 wk male (O102); heart fetal 28 wk male (O103); brain fetal 28 wk male (O104); gallbladder fetal 28 wk male (O106); small intestine fetal 28 wk male (O107); adipose tissue fetal 28 wk male (O108); ovary fetal 25 wk female (O109); uterus fetal 25 wk female (O110); testes fetal 28 wk male (O111); spleen fetal 28 wk male (O112); adult placenta 28 wk (O113); and tonsil inflamed, from 12 year old (X100).

V. Cloning of species counterparts of PrP2

Various strategies are used to obtain species counterparts of PrP2, preferably from other primates. One method employs cross hybridization using closely related species DNA probes. It may be useful to go into evolutionarily similar species as intermediate steps. For example, in situ hybridization methods were used to localize a mRNA for a chicken PrP protein. See, Harris, et al., (1993) Proc. Natl. Acad. Sci. USA 90:4309-4313. Additionally, a hamster PrP protein cDNA was used as a hybridization probe by Iwasaki, et al. (1992) Nucleic Acids Res. 20:4001-4007. Both techniques can be adopted here for use with PrP. Another method employs specific PCR primers based on the identification of blocks of similarity or difference between genes, e.g., areas of highly conserved or non-conserved polypeptide or nucleotide sequence. In addition, gene sequence databases may be screened for related sequences from other species.

VI. Production of mammalian PrP2 protein

An appropriate, e.g., GST, fusion construct is engineered for expression, e.g., in E. coli. For example, a mouse IGIF pGex plasmid is constructed and transformed into E. coli. Freshly transformed cells are grown, e.g., in LB medium containing 50 μg/ml ampicillin and induced with IPTG (Sigma, St. Louis, Mo.). After overnight induction, the bacteria are harvested and the pellets containing the prion8 protein are isolated. The pellets are homogenized, e.g., in TE buffer (50 mM Tris-base pH 8.0, 10 mM EDTA and 2 mM pefabloc) in 2 liters. This material is passed through a microfluidizer (Microfluidics, Newton, Mass.) three times. The fluidized supernatant is spun down on a Sorvall GS-3 rotor for 1 h at 13,000 rpm. The resulting supernatant containing PrP2 is filtered and passed over a glutathione-SEPHAROSE column equilibrated in 50 mM Tris-base pH 8.0. The fractions containing the PrP2-GST fusion are pooled and cleaved, e.g., with thrombin (Enzyme Research Laboratories, Inc., South Bend, Ind.). The cleaved pool is then passed over a Q-SEPHAROSE column equilibrated in 50 mM Tris-base. Fractions containing PrP2 are pooled and diluted in cold distilled H₂O, to lower the conductivity, and passed back over a fresh Q-Sepharose column, alone or in succession with an immunoaffinity antibody column. Fractions containing PrP2 are pooled, aliquoted, and stored in the −70° C. freezer.

Comparison of the CD spectrum with PrP2 may suggest that the protein is correctly folded. See Hazuda, et al. (1969) J. Biol. Chem. 264:1689-1693 and the teachings in U.S. Pat. No: 5,891,641 regarding PrP conformation. Such teachings can be applied without limit or undue effort to PrP2s.

VII. Preparation of antibodies specific for PrP2

Traditional methods of antibody preparation such as the following can be tried with the PrP2s of the invention: Inbred Balb/c mice are immunized intraperitoneally with recombinant forms of the protein, e.g., purified PrP2, or stable transfected NIH-3T3 cells. Animals are boosted at appropriate time points with PrP2, with or without additional adjuvant, to further stimulate antibody production. Serum is collected, or hybridomas produced with harvested spleens.

Alternatively, Balb/c mice are immunized with cells transformed with the gene or fragments thereof; endogenous cells, exogenous cells, or with isolated membranes enriched for expression of the antigen. Serum is collected at appropriate times, typically after numerous further administrations. Various gene therapy techniques may be useful, e.g., in producing protein in situ, for generating an immune response.

Monoclonal antibodies may be made. For example, splenocytes are fused with an appropriate fusion partner and hybridomas are selected in growth medium using standard procedures. Hybridoma supernatants are screened for the presence of antibodies that bind to the desired PrP2, e.g., by ELISA or other assay. Antibodies, which selectively recognize specific PrP2 embodiments, may also be selected or prepared.

In another method, synthetic peptides or purified PrP2 is presented to an immune system to generate monoclonal or polyclonal antibodies. See, e.g., Coligan (1991) Current Protocols in Immunology Wiley/Greene; and Harlow and Lane (1989) Antibodies: A Laboratory Manual Cold Spring Harbor Press. In appropriate situations, the binding reagent is either labeled as described above, e.g., fluorescence or otherwise, or immobilized to a substrate for panning methods. Nucleic acids may also be introduced into cells in an animal to produce the PrP2 antigen, which serves to elicit an immune response. See, e.g., Wang, et al. (1993) Proc. Natl. Acad. Sci. USA 90:4156-4160; Barry, et al. (1994) BioTechniques 16:616-619; and Xiang, et al. (1995) Immunity 2:129-135.

Alternatively, although the above procedures are typically effective, others have had difficulty in producing antibodies binding PrP proteins, e.g., disease forms of PrP. The difficulty relates, in part, to special qualities of the disease-related prion conformation. However, by following procedures described in U.S. Pat. Nos.: 5,846,533 and 5,792,901 (incorporated herein in their entirety) for generating PrP antibodies, one of ordinary skill in the art can adopt those techniques, without undue experimentation, to create antibodies to various alternative conformations of the PrP2s of the present invention.

When considering presenting an antigen to a mouse with the goal of obtaining specific monoclonal antibodies against a misfolded or aggregated form of a host protein, it is desirable to increase the definition of a subtle conformational difference. This can be achieved by immunizing an antigen knockout mouse that has not developed self-tolerance against the respective antigen. Furthermore, if conformational isoforms and/or oligomeric forms of a protein sequence are understood to exist in an equilibrium, high and pure amounts of recombinant protein may increase the likelihood that a particular population of protein conformation passes an antigenic threshold necessary to start an immunogenic response. Pulling out the monoclonal antibodies by correct screening is essential. Screening against the pure misfolded or aggregated protein is often complicated by its poor solubility and hence the ability to immobilize.

Another technique for creating monoclonal antibodies specific for the native, disease-associated isoform of the PrP prion is taught by Korth et al. (1999) Methods Enzymol. 309:106-122 and can be adapted here for use with PrP2 without requiring undue experimentation. Korth, et al., review the circumstances leading to the first monoclonal antibody against the disease-associated form of PrP. Using this analysis, Korth, et al., teach a reliable conformational screening protocol for successful immunization by immobilizing disease-associated PrP isoforms on nitrocellulose as a conformation-sensitive screening method, which permits freezing a PrP in a particular conformation, e.g., a distinguishable, disease-associated conformation.

An alternative strategy uses the nucleic acid-based immunization of Krasemann, et al. (1999) J. Biotechnol. 20,73:119-129. The method is based on nucleic acid injection into non tolerant PrP−/− mice. DNA or RNA coding for different human prion proteins including the mutated sequences associated with CJD, GSS, and FFI is injected into muscle tissue. Initially, the mice are inoculated with DNA-plasmids encoding PrP NP and boosted with either DNA, RNA, or recombinant Semliki Forest virus (SFV) particles that express PrP NP. After hybridoma preparation, different mAbs against PrP proteins are obtained and their binding behavior is analyzed by peptide-ELISA, Western blot, immunofluorescence, and immunoprecipitation. The resulting mAbs are directed against four different linear epitopes of PrP and may recognize discontinuous regions of the native prion protein. Immunization of non tolerant mice with DNA and live attenuated SF virus can induce a broad immune response eventually leading to the generation of a panel of mAbs for basic science as well as for diagnostic application.

IX. Production of fusion proteins with PrP2s

Various fusion constructs are made with PrP2s. A portion of the appropriate gene is fused to an epitope tag, e.g., a FLAG tag, or to a two hybrid system construct. See, e.g., Fields and Song (1989) Nature 340:245-246. Alternatively, the technique of Scott, et al. (1992) Chimeric prion protein expression-in cultured cells and transgenic mice Protein Sci. 1:986-997 can be adopted for use with PrP2.

The epitope tag may be used in an expression cloning procedure with detection with anti-FLAG antibodies to detect a binding partner, e.g., ligand for the respective PrP2. The two hybrid system may also be used to isolate proteins that specifically bind PrP2.

X. Structure activity relationship

Information on the criticality of particular residues may be determined using standard procedures and analysis. Standard mutagenesis analysis is performed, e.g., by generating many different PrP2 variants at determined positions and evaluating biological activities of the variants. This may be performed to the extent of determining positions that modify activity, or to focus on specific positions to determine the residues that can be substituted to either retain, block, or modulate biological activity.

Alternatively, analysis of natural variants or sequence comparisons (e.g., Tables 1 and 2) can indicate what positions tolerate natural mutations. This may result from population analysis of variation among individuals, or across strains or species. Samples from selected individuals are analyzed, e.g., by PCR analysis and sequencing. This allows evaluation of population polymorphisms.

XI. Isolation of a PrP2 ligand or binding compositions

A PrP2 can be used as a specific binding reagent to identify potential binding partners, by taking advantage of its specificity of binding, much like an antibody would be used. This method is similar to one employed to discover partners for a binding receptor that comprises a heterodimer of receptor subunits. The binding reagent, here, e.g., PrP2 is either labeled as described above, e.g., fluorescence or otherwise, or immobilized to a substrate for panning methods.

The binding composition is used to screen an expression library made from a cell line expressing a binding partner, e.g., ligand, preferably membrane associated. Standard staining techniques are used to detect or sort surface expressed ligand. Alternatively, surface-expressing, transformed cells are screened by panning. Screening of intracellular expression is performed by various staining or immunofluorescence procedures. See also McMahan, et al. (1991) EMBO J. 10:2821-2832.

For example, on day 0, precoat 2-chamber permanox slides with 1 ml per chamber of fibronectin, 10 ng/ml in PBS, for 30 min at room temperature. Rinse once with PBS. Then plate COS cells at 2-3×10⁵ cells per chamber in 1.5 ml of growth media. Incubate overnight at 37° C.

On day 1 for each sample, prepare 0.5 ml of a solution of 66 μg/ml DEAE-dextran, 66 μM chloroquine, and 4 μg DNA in serum free DME. For each set, a positive control and a negative mock are prepared. Rinse cells with serum free DME. Add the DNA solution and incubate 5 hr at 37° C. Remove the medium and add 0.5 ml 10% DMSO in DME for 2.5 min. Remove and wash once with DME. Add 1.5 ml growth medium and incubate overnight.

On day 2, change the medium. On days 3 or 4, the cells are fixed and stained. Rinse the cells twice with Hank's Buffered Saline Solution (HBSS) and fix in 4% paraformaldehyde (PFA)/glucose for 5 min. Wash 3× with HBSS. The slides may be stored at −80° C. after all liquid is removed. For each chamber, 0.5 ml incubations are performed as follows. Add HBSS/saponin (0.1%) with 32 μl/ml of 1 M NaN₃ for 20 min. Cells are then washed with HBSS/saponin 1×. Add appropriate PrP2 or PrP2/antibody complex to cells and incubate for 30 min. Wash cells twice with HBSS/saponin. If appropriate, add first antibody for 30 min. Add second antibody, e.g., Vector anti-mouse antibody, at 1/200 dilution, and incubate for 30 min. Prepare ELISA solution, e.g., Vector Elite ABC horseradish peroxidase solution, and preincubate for 30 min. Use, e.g., 1 drop of solution A (avidin) and 1 drop solution B (biotin) per 2.5 ml HBSS/saponin. Wash cells twice with HBSS/saponin. Add ABC HRP solution and incubate for 30 min. Wash cells twice with HBSS, second wash for 2 min. to close cells. Then add Vector diaminobenzoic acid (DAB) for 5 to 10 min. Use 2 drops of buffer plus 4 drops DAB and 2 drops of H₂O₂ per 5 ml of glass distilled water. Carefully remove chamber and rinse slide in water. Air dry for a few minutes, then add 1 drop of Crystal Mount and a cover slip. Bake for 5 min at 85-9° C.

Evaluate positive staining of pools and progressively subclone to isolation of single genes responsible for the binding.

Another strategy is to screen by panning. The cDNA is constructed as-described above. The ligand can be immobilized and used to immobilize expressing cells. Immobilization may be achieved by use of appropriate antibodies that recognize, e.g., a FLAG sequence of an PrP2 fusion construct, or by use of antibodies raised against the first antibodies. Recursive cycles of selection and amplification lead to enrichment of appropriate clones and eventual isolation of receptor expressing clones.

Phage expression libraries can be screened by mammalian PrP2s. Appropriate label techniques, e.g., anti-FLAG antibodies, will allow specific labeling of appropriate clones.

XI. Isolation of a peptide or protein that binds PrP2

One of skill in the art can easily adopt, without undue experimentation, a technique for identifying PrP binding proteins (U.S. Pat. No.: 5,679,530; incorporated herein) to identify anti-PrP2 binding compositions (e.g., anti-PrP2 binding proteins or polypeptides). The technique employs the concept of “complementary hydropathy” (Biro (1981) Medical Hypothesis 7:981), which is based on the observation that specific protein/protein interactions result between proteins encoded by a single gene but transcribed from both sense and anti-sense strands. Often, the resulting sense and “anti-sense” polypeptides specifically bind each other. The application of complementarity was used by Goldgaber (1991) Nature 352:291-292; and Moser, et al, (1993) Nature 362:213-214 to discover anti-PrP proteins. Hewinson, et al suggested that the complementary protein might function as a PrP receptor.

Employing this concept here, a peptide that is complementary to a polypeptide derived from PrP2 is synthesized using commonly known techniques (or as described herein). Particularly useful to determine the sequence of a PrP2 complementary peptide (e.g., one that binds PrP2) is to establish the sequence of a deduced anti-PrP2 ORF by using the PrP2 sequences provided herein.

The synthesized complementary PrP2 peptide is coupled to Keyhole limpet hemocyanin (KLH) to produce an immunogen. This immunogen is subsequently injected, intraperitoneally, into mice at two-week intervals, to a total of 100 ug of total protein. After the fourth injection, animals are bled, and titered against uncoupled complementary PrP2 peptide, via a standard ELISA. The resulting antibody is directed to and specifically binds complementary PrP2 peptide.

Isolated antibody can be used to determine complementary PrP2 expression in cell cultures. For example, a cell line suspected of harboring PrP2, such as a murine neuroblastoma cell line, is plated (2×10⁶ cells/well), e.g., in eight well tissue culture chamber slides. The cells are incubated overnight, after which the slides are washed, the cells fixed with 1% glutaraldehyde and then a 1:20 horse serum solution is added for 1 hour at 37° C. Following a wash with phosphate buffered saline (PBS), antiserum is added (approximately 2 h), followed by extensive washing then addition of second antibody for approximately 1 h, e.g., anti-IgG coupled to fluorescein isothiocyanate diluted 1:80 in Evan's blue. After further washing, the slides are mounted and examined to detect cells that stain for fluorescence suggesting the presence of complementary PrP2. Further analysis of positive and negative staining cells proceeds, e.g., as follows.

Positive staining and negative staining cells are cloned by limiting dilution. Living cells of each clone are surface labeled with e.g., ¹²⁵I, using a well-known lactoperoxidase method. Following labeling, cells are lysed and the resulting extracts are incubated with complementary PrP2 (determined as above) coupled overnight, with agitation to CNBr 4B SEPHAROSE beads (beaded agarose with wet bead diameter of 45-165 micrometers).

Following incubation, bound material (e.g., anything binding complementary PrP2 peptide) is separated by SDS-PAGE using a 40% stacking gel and a 7.5% resolving gel. Proteins are transferred to nitrocellulose filters (0.45 μm pore size), and stained with 0.5% PONCEAU S (3-hydroxy-4->2-sulfo-4-(4-sulfophenylazo)phenylazol-2,7-naphthalenedisulfonic acid) to determine and verify the extent of transfer. Results from negative and positive clones are run on separate lanes for comparison. A band for a protein/polypeptide of about the predicted appropriate molecular weight (kD) for a complementary PrP2 polypeptide (as seen in positive clones) should be absent in lanes representing negative clones and present in lanes representing positive clones. Further confirmation is carried out by Western blot techniques.

Cells from a positive clone are treated as above, except they are not labeled. In addition, as above, unlabeled protein extract is incubated with complementary PrP2 attached to SEPHAROSE beads (beaded agarose). Bound protein is eluted and applied to filters as described above. For Western blots, the filters are blocked with approximately 5% fat dry milk in PBS, and then incubated with either normal mouse serum or the antiserum described above, followed by goat anti-mouse biotin conjugated antibody. The biotin-conjugated antibody is added for approximately 1 h at room temperature. After extensive washing, the antibodies are developed, e.g., using a well-known ECL chemiluminescent system. The results are examined in separate lanes; e.g., with a control lane using normal serum, and an experimental lane using antiserum against anti-PrP2 complementary peptide as described above. Positive signaling suggests that antibody to PrP2 complementary peptide also recognizes a PrP2 band.

An isolated complementary PrP2 peptide can also be used to determine PrP2 in a sample. The technique involves contacting a sample with complementary PrP2 peptide to form complexes between it and a PrP2, followed by detection of the newly formed complex, e.g., using antiserum to complementary PrP2 peptide. An isolated complementary PrP2 peptide may be immobilized, e.g., on a bead, column glass tube wall, etc., but need not be. If not immobilized, complementary PrP2 peptide complexed to PrP2, e.g., formed in solution, can be determined by observing migration patterns on a gel. Further, the isolated protein may be labeled, e.g., with a chromophore, a radiolabel, an enzyme, or other standard label. Detecting presence of PrP2 in a sample may indicate presence of, or predisposition toward, a prion-type associated disorder, such as those described herein or in the art.

As noted, an isolated complementary PrP2 peptide may be used per se in diagnostic methods, or as an immunogen. In the latter case, an isolated complementary PrP2 peptide may be coupled to a carrier, such as keyhole limpet hemocyanin, bovine serum albumin, or any standard material used to haptenize small peptides. The resulting complex, comprising isolated complementary PrP2 peptide or the complementary PrP2 peptide per se, may be formulated in immunogenic compositions, such as with an adjuvant. As noted, antibodies produced following immunization with an isolated complementary PrP2 peptide can be used to detect cells carrying a PrP2 protein or polypeptide.

The identification and characterization of an anti-PrP2 binding protein can be used to identify PrP2 in samples, thus providing a method of screening and/or diagnosis, especially when other symptoms characteristic of a prion associated disorder are observed. In view of the prevalence of prion associated disorders in livestock, e.g., there are both human and veterinary uses for the PrP2s of this invention.

All citations herein are incorporated herein by reference to the same extent as if each individual publication, patent application, or patent was specifically and individually indicated to be incorporated by reference. All citations are incorporated in their entirety, including all figures, pictures, drawings, tables, forms, diagrams without exclusion.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not-to be limited in any manner by the specific embodiments which have been presented herein by way of example.

What is claimed is:

1. An isolated, or recombinant PrP2 comprising a mature polypeptide sequence of SEQ ID NO: 2, 4, or 6, or antigenic fragments thereof at least 12 contiguous amino acid residues in length.

2. The polypeptide of claim 1, wherein:
a) said contiguous amino acid residues are at least 15;
b) said contiguous amino acid residues are at least 22;
c) said contiguous amino acid residues are at least 25;
d) said contiguous amino acid residues are at least 30;
e) said contiguous amino acid residues are at least 40;
f) said contiguous amino acid residues are at least 50;
g) said contiguous amino acid residues are at least 75; or
d) said contiguous amino acid residues are at least 100.

3. The polypeptide of claim 2, which:
a) comprises a fusion polypeptide;
b) comprises sequence of SEQ ID NO: 2, 4, or 6;
c) specifically binds antibody raised against a polypeptide comprising said PrP2 polypeptide sequence; or
d) said polypeptide:
i) is from a warm blooded animal such as a primate or a rodent;
ii) comprises at least one polypeptide segment of SEQ ID NO: 2, 4, or 6;
iii) comprises a plurality of polypeptide segments of SEQ ID NO: 2, 4, or 6;
iv) is a natural allelic variant of a primate or rodent PrP2;
v) has a length at least about 120 amino acids;
vi) exhibits at least two non-overlapping epitopes that are specific for a primate or rodent PrP2;
vii) exhibits identity over a length of at least 35 amino acids to a primate or rodent PrP2;
viii) comprises heterologous amino acid sequence;
ix) is encoded by polynucleotide sequence operably linked in an expression vector;
x) is a synthetic polypeptide;
xi) is a full length mature polypeptide of SEQ ID NO: 2, 4, or 6;
xii) is attached to a solid substrate;
xiii) is conjugated to another chemical moiety;
xiv) is a 5-fold or less substitution from natural PrP2 sequence; or
xv) is a deletion or insertion variant from a natural PrP2 sequence.

4. A composition comprising:
a) a sterile polypeptide of claim 1;
b) said polypeptide of claim 1 and a carrier, wherein said carrier is:
i) an aqueous compound, including water, saline, and/or buffer; and/or
ii) formulated for oral, rectal, nasal, topical, or parenteral administration.

5. The fusion polypeptide of claim 3, comprising:
a) mature polypeptide sequence of SEQ ID NO: 2, 4, or 6;
b) a detection or purification tag, including a FLAG, His6, or Ig sequence; or
c) sequence of another PrP polypeptide.

6. A kit comprising a polypeptide of claim 1, and:
a) a compartment comprising said polypeptide; and/or
b) instructions for use or disposal of reagents in said kit.

7. A binding compound comprising an antigen binding site from an antibody that specifically binds a polypeptide of claim 1, wherein:
a) said polypeptide is from a primate or rodent;
b) said binding compound is an Fv, Fab, or Fab2 fragment;
c) said binding compound is conjugated to another chemical moiety; or
d) said antibody:
i) is raised against a polypeptide sequence of SEQ ID NO: 2, 4, or 6;
ii) is raised against a mature primate or rodent PrP2;
iii) is raised to a purified human PrP2;
iv) is raised to a purified mouse PrP2;
v) is immunoselected;
vi) is a polyclonal antibody;
vii) binds to a denatured or soluble PrP2;
viii) exhibits a Kd to antigen of at least 30 μM;
ix) is attached to a solid substrate, including a bead or plastic membrane;
x) binds an abnormal conformational form of PrP2
xi) is in a sterile composition; or
xii) is detectably labeled, including a radioactive or fluorescent label.

8. A kit comprising said binding compound of claim 7, and:
a) a compartment comprising said binding compound; and/or
b) instructions for use or disposal of reagents in said kit.

9. A method of:
a) making an antibody of claim 7, comprising immunizing an immune system with an immunogenic amount of a rodent or primate PrP2 polypeptide thereby causing said antibody to be produced; or
b) producing an antigen:antibody complex, comprising contacting a rodent or primate PrP2 protein or polypeptide with an antibody of claim 7, thereby allowing said complex to form.

10. A composition comprising:
a) a sterile binding compound of claim 7, or
b) said binding compound of claim 7 and a carrier, wherein said carrier is:
i) an aqueous compound, including water, saline, and/or buffer; and/or
ii) formulated for oral, rectal, nasal, topical, or parenteral administration.

11. An isolated, or recombinant polynucleotide encoding a polypeptide of claim 1, wherein:
a) said PrP2 is from a mammal; or
b) said polynucleotide:
i) encodes an antigenic sequence of at least 12 contiguous amino residues from SEQ ID NO: 2, 4, or 6;
ii) encodes a plurality of antigenic peptide sequences of SEQ ID NO: 2, 4, or 6;
iii) exhibits identity to a natural cDNA encoding said segment;
iv) is operably linked in an expression vector;
v) further comprises an origin of replication;
vi) is from a natural source;
vii) comprises a detectable label;
viii) comprises synthetic nucleotide sequence;
ix) is less than 6 kb, preferably less than 3 kb;
x) is from a mammal, including a primate, such as a human;
xi) comprises a natural full length coding sequence;
xii) comprises heterologous sequence;
xiii) is a hybridization probe for a gene encoding said PrP2;
xiv) comprises a plurality of non-overlapping segments of at least 15 nucleotides from SEQ ID NO: 1, 3, 5, or 13; or
xv) is a PCR primer, PCR product, or mutagenesis primer.

12. A cell transfected or transformed with said polynucleotide of claim 11.

13. The cell of claim 12, wherein said cell is:
a) a prokaryotic cell;
b) a eukaryotic cell;
c) a bacterial cell;
d) a yeast cell;
e) an insect cell;
f) a mammalian cell;
g) a mouse cell;
h) a primate cell; or
i) a human cell.

14. A kit comprising said polynucleotide of claim 11, and:
a) a compartment comprising said polynucleotide;
b) a compartment further comprising a primate or rodent PrP2 polypeptide; and/or
c) instructions for use or disposal of reagents in said kit.

15. A method of:
a) making a polypeptide, comprising expressing said polynucleotide of claim 11, thereby producing said polypeptide; or
b) making a polypeptide, comprising expressing said polynucleotide of claim 11 in a cell of claim 12; or
b) making a duplex polynucleotide, comprising contacting said polynucleotide of claim 11 with a complementary polynucleotide to form said duplex.

16. A polynucleotide that:
a) hybridizes under wash conditions of 40° C. and less than 2M salt to a nucleic acid with a sequence of SEQ ID NO: 1, 3, or 5; or
b) exhibits identity over a stretch of at least about 30 nucleotides to a primate or rodent PrP2 of SEQ ID NO: 1, 3, 5, or 13; or
c) encodes a polypeptide comprising at least 14 contiguous amino acids of a primate or rodent PrP2 polypeptide of SEQ ID NO: 2, 4, or 6.

17. The polynucleotide of claim 16, wherein:
a) said wash conditions are at 55° C. and/or 500 mM salt; or
b) said stretch is at least 55 nucleotides.

18. The polynucleotide of claim 17, wherein:
a) said wash conditions are at 65° C. and/or 150 mM salt; or
b) said stretch is at least 75 nucleotides.

19. A method of modulating physiology or development of a cell or tissue culture cell comprising contacting said cell with an agonist or antagonist of a primate or rodent PrP2.

20. The method of claim 19, wherein said cell is transformed with a polynucleotide encoding either a rodent or primate PrP2.
